(19) United States
(12) Patent Application Publication
(10) Pub. No.: US 2022/0056491 A1
(43) Pub. Date: Feb. 24, 2022

(54) MICROBIAL CONVERSION OF OILS AND FATS TO LIPID-DERIVED HIGH-VALUE PRODUCTS

(71) Applicant: The University of Massachusetts, Boston, MA (US)

(72) Inventors: Dongming Xie, Dracut, MA (US); Ya-Hue Valerie Soong, Nashua, NH (US); Na Liu, Lowell, MA (US); Andrew Thomas Olson, Winchester, MA (US); Hsi-Wu Wong, Burlington, MA (US)

(21) Appl. No.: 17/341,767

(22) Filed: Jun. 8, 2021

Related U.S. Application Data
(60) Provisional application No. 63/037,151, filed on Jun. 10, 2020.

(51) Int. Cl. C12P 7/64; C12N 9/02; C12N 9/10; C12N 1/16; C12N 1/20; C12N 9/20

(52) U.S. Cl. CPC C12P 7/6436; C12N 9/0008; C12Y 102/01084; C12N 9/1029; C12N 2511/00; C12N 1/16; C12N 1/20; C12N 9/20; C12P 7/6427; C12Y 203/01075

(57) ABSTRACT

A method of directly microbially converting a plant oil, an animal fat, free fatty acid, or a combination thereof to wax esters includes growing a yeast or bacterial strain in a medium comprising the plant oil, the animal fat, the free fatty acid, or combination thereof, under conditions suitable to produce the wax esters, wherein the yeast or bacterial strain is engineered to express a FAR gene encoding fatty acid alcohol reductase and a WS gene encoding a wax ester synthase, and optionally isolating the produced wax esters. Similar methods of directly microbially converting a plant oil, an animal fat, free fatty acid, or a combination thereof to omega-3 fatty acids by growing a microorganism in a medium comprising the plant oil, the animal fat, the free fatty acid, or combination thereof, under conditions suitable to produce omega-3 fatty acids are also described.

### CROSS-REFERENCE TO RELATED APPLICATIONS

This application claims priority to U.S. Provisional Application 63/037,151 filed on Jun. 10, 2020, which is incorporated herein by reference in its entirety.

### FIELD OF THE DISCLOSURE

The present disclosure is related to methods for directly preparing wax esters and omega-3 fatty acids using yeast and/or bacterial strains.

### BACKGROUND

Plant oils (or vegetable oils) and animal fats are important agricultural commodities from oil crops (e.g., palm, soybean, rape seed, etc.) and the rendered animal fat industry, with an annual production of approximately 20 million tons in the US. This is about twice as much as the total US sugar production according to the United States Department of Agriculture-Foreign Agriculture Service (USDA-FAS, 2019). While sugars are widely used in the biotechnology industry to make fuels, chemicals, and value-added bioproducts, oils and fats are primarily used for food, feed, or nutritional applications with low or limited economic value. In addition, millions of tons of waste cooking oils and fats are being generated every year from primary food applications. While some waste cooking oils and fats are used for biodiesel, bioplastics, or other chemical production, a significant portion of waste oils and fats are released to the environment without appropriate treatment and causes serious pollution. Disposal of waste oils/fats is a big concern due to the uncertain biodiesel market and the pollution caused by the uncontrolled release to environment.

What is needed are new processes to convert excess or waste fats and oils into high value products.

### BRIEF SUMMARY

In an aspect, a method of directly microbially converting a plant oil, an animal fat, free fatty acid, or a combination thereof to wax esters comprises growing a bacterial strain in a medium comprising the plant oil, the animal fat, or a combination thereof, under conditions suitable to produce the wax esters, wherein the bacterial strain is engineered to express a FAR gene encoding fatty acid alcohol reductase and a WS gene encoding a wax ester synthase, or growing a yeast strain in a medium comprising the plant oil, the animal fat, the free fatty acid, or a combination thereof, under conditions suitable to produce the wax esters, wherein the yeast strain is engineered to express a FAR gene encoding fatty acid alcohol reductase and a WS gene encoding a wax ester synthase; and optionally isolating the produced wax esters.

In another aspect, a method of directly microbially converting a plant oil, an animal fat, a fatty acid, or a combination thereof, to omega-3 fatty acids comprises growing a microorganism that produces omega-3 fatty acids (e.g., a metabolically engineered Y. lipolytica strain Y8412 (ATCC #PTA-10026)) in a medium comprising the plant oil, the animal fat, the fatty acid, optionally glucose and optionally glycerol, under conditions suitable to produce the omega-3 fatty acids, and optionally isolating the produced omega-3 fatty acids.

### BRIEF DESCRIPTION OF THE DRAWINGS

FIG. 1 illustrates an aspect of a biomanufacturing platform to make high-value products such as wax esters, omega-3 fatty acids, long-chain diacids, and carotenoids from plant oils or animal fats.

FIG. 2 illustrates a comparison between the metabolic engineering strategies for biosynthesis of wax esters from oils/fats (A—present method) and from glucose (B—prior art method).

FIG. 3 shows a comparison between the metabolic engineering strategies for biosynthesis of omega-3 EPA from oils/fats (A—present method) and from glucose (B—prior art method).

FIG. 4 shows an overview of metabolic engineering strategy in Y. lipolytica for production of wax esters from either glucose or fatty acids. Fatty acyl-CoA was converted into fatty alcohol by fatty acyl-CoA reductase (FAR). Fatty alcohols and an acyl-CoA are esterified to form wax ester by wax ester synthase (WS). Gray box: wax ester biosynthesis pathway.

FIG. 5 shows a genome engineering strategy for wax ester synthesis in yeast Y. lipolytica. Procedure of FAR-WS-URA3 linear cassette extraction from restriction enzyme digestions: Linear FAR-WS-URA3 gene was released from the Xbal and EcoRI cleavages. Restriction double digestion was confirmed via agarose gel electrophoresis and the ideal DNA size of linearized FAR-WS-URA3 was 6,562 bp.

FIG. 6 shows yeast colony screening via PCR. Linear FAR-WS-URA3 structure was confirmed and identified via three primer sets of PCR. Genomic DNA were extracted from twelve yeast colonies that transformed with linearized FAR-WS-URA3. FAR-FP forward primer and WS-RP reverse primer were selected to roughly screen FAR-WS-URA3 cassette (FAR connecting with WS gene: 3,732 bp) existing in the genome. The coding sequence (CDS) size of fatty alcohol reductase (FAR: 1,539 bp) and wax synthase (WS: 1,392 bp) gene were analyzed via FAR-FP/FAR-RP and WS-FP/WS-RP as primer sets, respectively.

FIG. 7 shows a microscopy study of the engineered wax ester producing strain VSWE1 in a shaking flask. The strain VSWE1 was fed with glucose, soybean oil, or waste cooking oil (WCO) as substrate in flask culture. The cells from the 120-h samples were examined under 1,000× oil immersion objective microscope. LB represents lipid bodies.

FIG. 8A-C show a comparison between cell growth of the Y. lipolytica strains ATCC20362, VSWE1, VSWE2, VSWE3, VSWE4, and VSWE5 in shake flask experiments with YD (glucose), YO (soybean oil), and YWCO (waste cooking oil) medium.

FIG. 9A-C show a comparison between wax ester production by the Y. lipolytica VSWE1, VSWE2, VSWE3, VSWE4, and VSWE5 in shake flask experiments with YD (glucose), YO (soybean oil), and YWCO (waste cooking oil) medium.

FIG. 10 shows a comparison between the specific wax ester production by the Y. lipolytica VSWE1, VSWE2, VSWE3, VSWE4, and VSWE5 at 120 h in shake flask experiments with YD (glucose), YO (soybean oil), and YWCO (waste cooking oil) medium.

FIG. 11 shows a comparison between wax ester production by the Y. lipolytica VSWE1 in shake flask experiments with YD (glucose), YO (soybean oil), and YWCO (waste cooking oil) medium.

FIG. 12. illustrates construction of three new E. coli strains for wax ester production. Strain BL21(DE3)/pFAR/pWS contains two separate plasmids. The plasmid pFAR and pWS were developed by cloning the FAR and WS genes into the expression vector pET21b(+) and pET28a(+), respectively. Strain BL211(DE3)/pFAR-WS contains the plasmid pFAR-WS, which has both FAR and WS genes introduced into the pET21b(+) with each gene under an individual T7 promoter. Strain BL21(DE3)/p(FAR-WS) contains the plasmid p(FAR-WS), which has both FAR and WS genes fused together under a shared T7 promoter.

FIG. 13 shows identification of enzyme expression by one-dimensional PAGE analysis. The FAR and WS genes containing polyhistidine tag were extracted from engineered bacterial cells and purified by immobilized metal affinity chromatography. The size and molecular weight of fatty acyl-CoA reductase and wax ester synthase were analyzed via one-dimensional polyacrylamide gel electrophoresis. The molecular weight of fatty acyl-CoA reductase was 57 kDa, wax ester synthase was 54 kDa and FAR-WS fused enzyme was about 110 kDa. Panels show, from left to right, BL21(DE3) w/pFAR; w/pWS; w/pFAR and pWS; w/pFAR-WS: and w/p(FAR-WS).

FIGS. 14A and B show a wax ester profile of engineered E. Coli BL21(DE3) strains in a shaking flask. 14A shows the wax ster titer (g/LO at 30 h, and 14B shows the wax ester specific titer (g/g) at 30 hr. E. coli BL21(DE3) was transformed with pFAR and pWS, pFAR-WS or p(FAR-WS) plasmid, whose data are shown in green, yellow, and red bars, respectively. Engineered bacterial strains were cultured in shaking flask using glucose and glycerol, C18:1 free fatty acid (oleic acid), lipase-digested soybean oil, or waste cooking oil (WCO) as carbon sources. Specific productivity of wax esters was determined by the ratio of wax ester titer to dry cell weight. All sample were collected at 30 hours.

FIGS. 15A and B show a comparison between fermentation results of the E. coli BL21(DE3)/p(FAR-WS) with glucose, glucose+oleic acid, and glucose+waste cooking oil+lipase. (A) dry cells weight; (B) wax esters titer. DCW: dry cells weight, Glc: glucose, OLA: oleic acid, WCO: waste cooking oil.

FIG. 16A-C show a comparison between the wax ester profiles from the fermentation of the E. coli BL21(DE3)/p(FAR-WS) with glucose, glucose+oleic acid, and glucose+waste cooking oil+lipase. Glc: glucose, OLA: oleic acid, WCO: waste cooking oil.

FIG. 17A-F show a comparison between Y8412 fermentation results with feeding of four different combinations of glucose, WCO, HWCO, and lipase. (A) Dry cells weight (DCW); (B) Total intracellular lipid titer; (C)-(F) Total omega-3 EPA titer and composition under the conditions of feeding four different combinations of glucose, WCO, HWCO, and lipase. Glc: glucose, WCO: waste cooking oil, HWCO: hydrolyzed waste cooking oil. TAG EPA: trieicosapentaenoic or triacylglyceride of EPA, free EPA: free fatty acid of EPA. When WCO/HWCO was fed in three injections into the 1-L bioreactor containing 700 mL fermentation medium, the feeding profile was 10 mL at 36 h, 5 mL at 48 h, and 5 mL at 72 h, respectively. For the feeding case of Glc+WCO+lipase, lipase was co-injected with WCO at a ratio of 0.02 g lipase/mL WCO.

FIG. 18A-D show a comparison between fermentation results of strain Y8412 by co-feeding of WCO and lipase with either glucose or glycerol: (A) results of dry cells weight (DCW); (B) total intracellular lipid titer by acid-catalyzed transesterification and esterification method; (C)-(D) total omega-3 EPA titer and omega-3 EPA composition at different time points in 1-L fed-batch fermentation. DCW: dry cells weight, Glc: glucose, Gly: glycerol, HWCO: hydrolyzed waste cooking oil, WCO: waste cooking oil, TAG EPA: trieicosapentaenoic or triacylglyceride of EPA, free EPA: free fatty acid of EPA

The above-described and other features will be appreciated and understood by those skilled in the art from the following detailed description, drawings, and appended claims.

### DETAILED DESCRIPTION

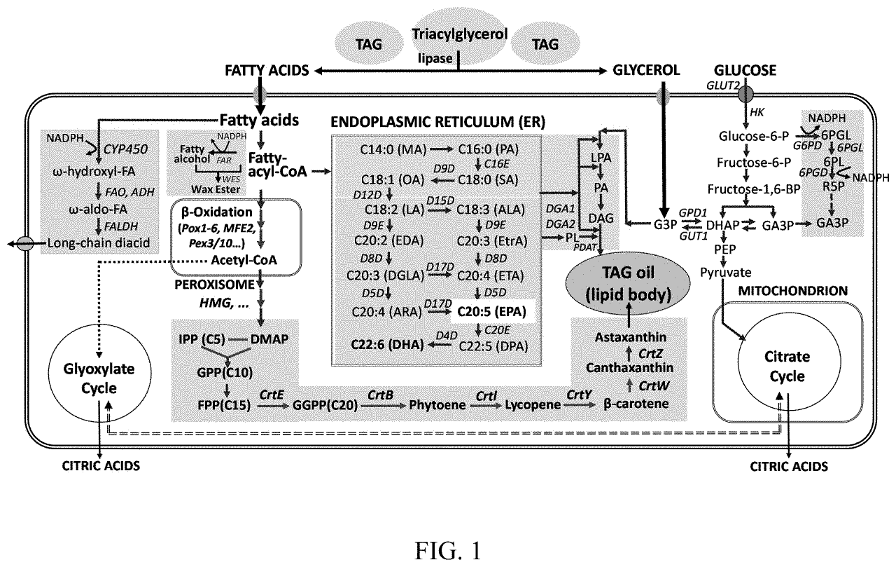

To significantly increase the economic value of oils and fats, described herein is a new biomanufacturing technology platform that uses engineered bacteria or yeasts to directly convert plant oils, animal fats or fatty acids derived therefrom into high-value products such as wax esters for cosmetics and high-performance lubricants, and omega-3 fatty acids for brain development and heart health. The market for these products is estimated at approximately 10 billion US dollars. Other high-value or value-added products include long-chain diacids for high-performance nylons, carotenoids as antioxidants for nutraceuticals and pharmaceuticals, PHA (polyhydroxyalkanoate) for biodegradable plastics, and the like (FIG. 1).

### Wax Ester Production

Currently, the major sources of wax esters are from the Jojoba plant and Sperm whales. Due to the hunting ban of Sperm whales and the harsh requirements of agricultural systems for Jojoba, wax esters are in short supply, which has led to the exploration of microbial, enzymatic, and chemical technology routes to increase current production levels. Most studies of microbial synthesis of wax esters use glucose as the starting material to synthesize wax esters, which requires glucose to be first converted to fatty acids and then further converted to wax esters. This process can be achieved by metabolic engineering through improving the existing fatty acid pathway and the building wax ester synthesis pathway. Since the conversion yield from glucose to fatty acids in usually low (<0.25 g/g), the overall production titer, rate and yield of wax esters from glucose is unfortunately not economically attractive.

Recently, an enzymatic route for wax ester production has been explored. This technology uses lipases to first convert microbial or vegetable oils into free fatty acids, which are then reacted with fatty alcohols to make wax esters through a transesterification reaction. The fatty alcohols in this process need to be either purchased from other sources or prepared by hydrogenolysis of fatty acids, which make this technology less competitive as compared to any one-step synthesis technology that directly converts oils into wax esters.

In addition to the enzymatic technology, chemical modifications using a base catalyst, peroxide and carboxylic acid, additional alcohols can be used to make esters as biolubricants. However, since additional short-chain alcohols are used to make the esters, the quality is not comparable to the wax esters that are formed from long-chain alcohols and fatty acids.

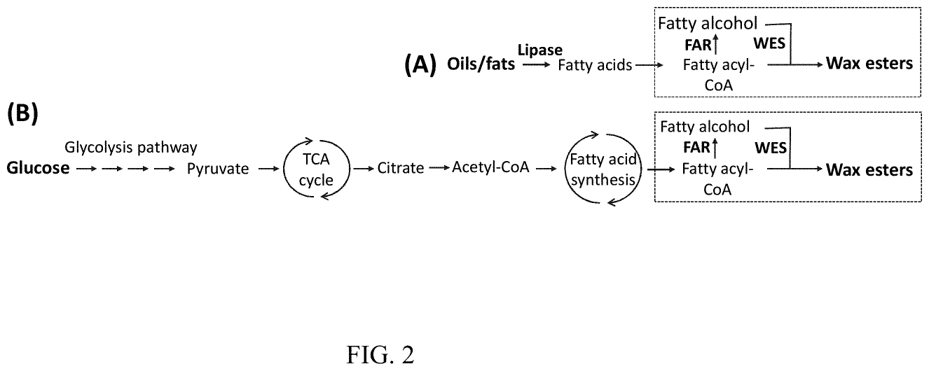

In an aspect, described herein is a method to produce wax esters from oils/fats by direct microbial conversion (FIG. 2), where both E. coli and yeast Y. lipolytica strains were engineered to express the two key genes required for biosynthesis of wax ester synthesis: FAR encoding fatty alcohol reductase from the bacterium M. hydrocarbonoclasticus and WS encoding wax ester synthase from A. calcoaceticus. Oils/fats are decomposed to free fatty acids by lipase, which can be produced by the Y. lipolytica yeast. Fatty acyl-CoAs are formed when the fatty acids are introduced inside the cells. FAR catalyzes the reaction of converting fatty acids into fatty alcohols, which then react with fatty acyl-CoAs to synthesize wax esters with the catalysis by WS.

The biosynthesis pathway from fatty acids to wax esters has been studied previously. However, previous research on biosynthesis of wax esters has been focused on using sugars (mainly glucose) as the starting materials, which need to be first converted into intracellular fatty acids, and then the formed fatty acids are further converted to wax esters. Since the conversion yield from glucose to fatty acids in usually low (<0.25 g/g), the overall production titer, rate and yield of wax esters from glucose is not economically attractive. The experimental data presented herein demonstrates that wax ester production was improved by about 70 fold in the engineered yeast Y. lipolytica (see Table 1) and 2-8 fold in the engineered bacterium E. coli when waste cooking oils was used as the substrate to completely or partially replace glucose.

TABLE 1
PRELIMINARY RESULTS OF PRODUCTION OF
HIGH-VALUE PRODUCTS FROM GLUCOSE AND
WASTE COOKING OILS (WCO) BY THE INITIALLY
ENGINEERED Y. LIPOLYTICA YEAST

| | Product titers (g/L) from different substrates | | | |
|---|---|---|---|---|
| Product | Glucose | | WCO or FFA | Glucose + WCO |
| Wax Esters | 0.11 | g/L | 7.7 g/L | N/A |
| Omega-3 EPA | 5.2 | g/L | N/A | 7.8 g/L |

Wax esters are widely distributed natural compounds that are found in highly evolved plants, algae, microorganisms, insects and mammals. Naturally occurring waxes, consisting of fatty acids esterified to long chain alcohols, are a group of highly hydrophobic neutral lipids, but they are structurally diverse. The physical properties and applications of wax esters are varied due to different chain lengths of the fatty acid and the fatty alcohol components as well as the degree of the unsaturation that affect melting temperature, oxidation stability and pressure stability. Wax esters have a variety of biological functions that provide the protective coating on the surface so that they are resistant to dehydration, UV light and pathogens. Wax esters are used commercially to serve in a wide range of applications, such as cosmetics, printing inks, lubricants, coatings, pharmaceutical and the food industry. The establishment of efficient expression platform will be highly expected to advance the economic feasibility of wax esters from low-cost substrates, especially from plant oils.

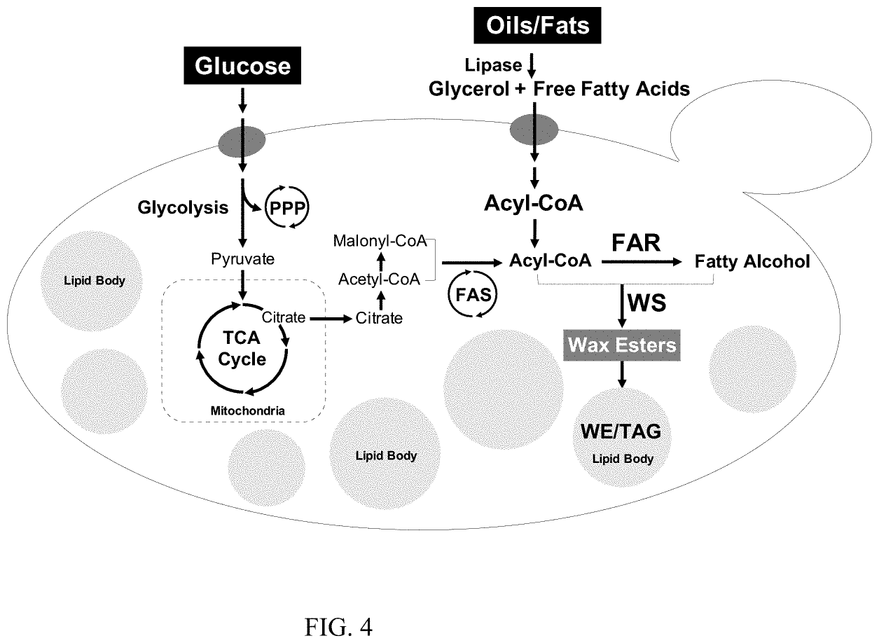

More specifically, as described herein, new Y. lipolytica yeast and E. coli strains were constructed for biosynthesis of wax esters from sugars and/or fatty acid substrates. The oleaginous yeast Y. lipolytica is one of the extensively well-characterized and most commonly used microorganisms in the biotechnology industry. Y. lipolytica is considered as an ideal host for further engineering for large-scale wax ester production due to the capability of efficient fatty acid utilization and high-level triacylglyceride (TAG) accumulation. Introduction of a heterologous metabolic pathway of wax ester into Y. lipolytica is shown in FIG. 4. Two genes, FAR from Marinobacter hydrocarbonoclasticus strain VT8 encoding the fatty acyl coenzyme A and WS from Acinetobacter calcoaceticus strain ADP1 encoding the wax ester synthase/acyl-CoA: diacylglycerol acyltransferase (WS/DGAT), are required for wax ester biosynthesis. The two genes were integrated into the Y. lipolytica's chromosome by transformation of their linear DNA fragments under the control of the strong TEF1 promoter.

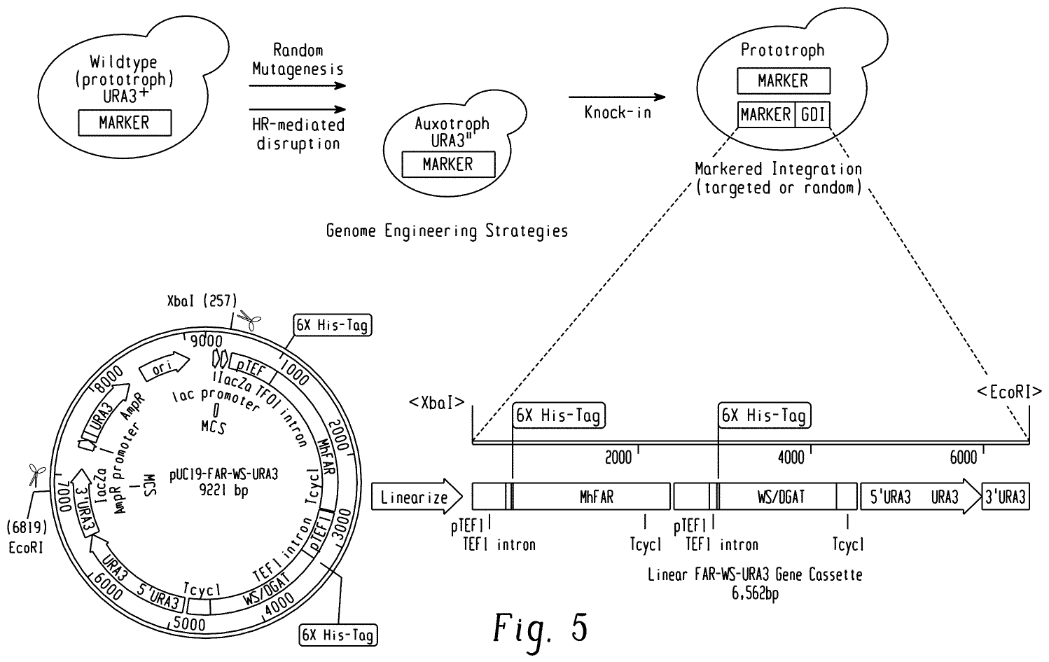

In comparison with yeast Y. lipolytica, the E. coli bacterial system, especially using a plasmid, is widely adopted as the host strain for overexpression of target genes due to its rapid production rates, easy gene manipulation, short and inexpensive culture and large quantities of target production. To establish a suitable inducible gene expression system, the E. coli BL21(DE3) as a T7 RNA polymerase system was selected for overexpression of FAR and WS gene under the control of the T7 promoter (FIG. 5), which is turned on after the inducer IPTG is added during the fermentation. In addition, the two genes were either individually expressed with each controlled under the T7 promoter or fused together under one T7 promoter. The development of bifunctional fusion enzyme was inspired for promotion of substrate channeling to address the limitation of the intracellular enzymes distance and active site orientation.

In an aspect, a method of directly microbially converting a plant oil, an animal fat, free fatty acid, or a combination thereof to wax esters comprises growing a yeast or bacterial strain in a medium comprising the plant oil, the animal fat, the free fatty acid, or combination thereof, under conditions suitable to produce the wax esters, wherein the yeast or bacterial strain is engineered to express a FAR gene encoding fatty acid alcohol reductase and a WS gene encoding a wax ester synthase, and optionally isolating the produced wax esters.

In an aspect, the medium is glucose-free.

Exemplary plant oils include, but are not limited to palm oil, soybean oil, corn oil, rapeseed oil, peanut oil, sunflower oil, coconut oil, cotton seed oil, olive oil, and combinations thereof. Exemplary animal fats include, but are not limited to beef fat, chicken fat, pork fat, fish fat or oil, and combinations thereof.

Fatty acids can be derived from the foregoing oils and fats using a hydrolysis process with the catalysis of lipases. Exemplary fatty acids include, but are not limited to capric acid (C10:0), undecylic acid (C11:0), lauric acid (C12:0), tridecylic acid (C13:0), myristic acid (C14:0), pentadecylic acid (C15:0), palmitic acid (C16:0), palmitoleic acid (C16:1), margaric acid (C17:0), stearic acid (C18:0), oleic acid (C18:1), linoleic acid (C18:2), linolelaidic acid (C18:2), γ-linolenic acid (C18:3), α-linolenic acid (C18:3), nonadecylic acid (C19:0), arachidic acid (C20:0), and combinations thereof.

The plant oils, animal fats, and free fatty acids can be obtained from any available agricultural commodity on the market (as fresh or unused oils/fats) or from byproducts and wastes that are generated from food processing (such as waste cooking oils or used cooking oils from restaurants), biodiesel production, and biomass treatment processes. Waste cooking oil (WCO or used cooking oil UCO) refers to vegetable oils or animal fats recycled and collected from food processing, food preparation, and cooking processes (e.g., the used cooking oil after preparing French fries and frying chickens).

In an aspect, the medium comprises a lipase. Lipases can help hydrolyze the triacylglyceride oils/fats to into free fatty acids or help accelerate the hydrolysis process, that is, to help decompose the plant oil or animal fats into free fatty acids. The lipase can be produced by the yeast or bacterial strain, provided in the medium, fed during the growth process, or a combination thereof. Lipase can be made by many live cells. For example, the most commonly animal lipase is produced from pancreatic gland. With regard to plant, papaya latex, oat seed land castor seed can serve source of lipase. However, most commercially produced lipases have been produced from fungi (such as Yarrowia lipolytica, Candida lipolytica, Geotrichum candidum and Penicillium roqueforti) and bacteria (such as Bacillus thermocatenulatus, Pseudomonas and Moraxella sp., Pyrococcus furiosus and Thermotoga sp., Pseudomonas fluorescens, Bacillus sp., B. coagulans and B. cereus, B. stearothermophilus, Geotrichum sp. and Aeromonas sobria, and P. aeruginosa) due to the high yield and low production cost. Exemplary producers of microbial lipases include Novozymes (Denmark), DSM (Netherlands), Chr. Hansen (Denmark), Amano Enzymes (Japan), Associated British Foods (UK), DuPont (US), and International Flavors & Fragrances (US)

The yeast or bacterial strain is engineered to express a FAR gene encoding fatty acid alcohol reductase and a WS gene encoding a wax ester synthase. In an aspect, the FAR gene expresses MhFAR from M. hydrocarbonoclasticus (also known as M. aqueaolei) (Gene Accession Number: WP_011785687.1; SEQ ID NO: 1), AmFAR from the honeybee (Apis melhfera) (Gene Accession Number: NM_001193290.1; SEQ ID NO: 2), HsFAR from Homo sapiens brain tissue (Gene Accession Number: NX_Q8WVX9.1; SEQ ID NO: 3), AtFAR5 from Arabidopsis thaliana (Gene Accession Number: Q39152; SEQ ID NO: 4), or MmFAR from house mouse (Mus musculus) (Gene Accession Number: Q922J9; SEQ ID NO: 5). In an aspect, the WS gene is from Jojoba Simmondsia chinensis (Gene Accession Number: AF149919; SEQ ID NO: 6) and microorganisms including but not limited to A. calcoaceticus or A. baylyi (Gene Accession Number: CAG67733.1; SEQ ID NO: 7), M. hydrocarbonoclasticus (Gene Accession Number: EF219376.1; SEQ ID NO: 8), R. opacus (Gene Accession Number: OPAG_07212; SEQ ID NO: 9), and P. arcticus (Gene Accession Number: Q4FV62; SEQ ID NO: 10).

Exemplary oleaginous yeast strains for use in the methods include, but are not limited to, Y. lipolytica, Rhodosporidium toruloides, Lipomyces starkey, Rhodotorula glutinis, Trichosporon fermentans, and Cryptococcus curvatus.

Exemplary bacterial strains for use in the methods include, but are not limited to, E. coli, Bacillus subtilis, Streptococcus pneumonia, and other bacterial strains that can metabolize a fatty acid substrate. In a specific aspect, the bacteria is E. coli and a T7 RNA polymerase system is used for expression of the FAR and WS genes.

Also included herein is a Yarrowia lipolytica ATCC20362 strain engineered to express a FAR gene encoding fatty acid alcohol reductase and a WS gene encoding a wax ester synthase, such as Yarrowia lipolytica strains VSWE1-5.

Further included is an E. coli BL21(DE3) strain engineered to express a FAR gene encoding fatty acid alcohol reductase and a WS gene encoding a wax ester synthase.

### Omega-3 Fatty Acid Production

Omega-3 fatty acids refer to the long-chain polyunsaturated fatty acids (LCPUFA) with the first C═C double bond at the n-3 (or omega-3) position, i.e., the third carbon from the methyl end of the carbon chain. Eicosapentaenoic acid (C20:5; EPA) and docosahexaenoic acid (C22:6; DHA) are the two major omega-3 fatty acids that are widely studied and have been demonstrated great health benefits in improving heart health, immune function, mental health, and infant cognitive development. In human nutrition, the omega-3 EPA and DHA are largely obtained from the diet, especially cold-water oceanic fishes, such as wild salmon and Pacific sardine that can accumulate significant amounts of EPA and DHA by eating microalgae cells. Currently, fish oil is the main source of EPA and DHA in the human diet, but its availability and sustainability have been questioned due to over-fishing and probable contamination in the ocean environment. To overcome this limitation, the biotechnology industry has started to produce DHA and/or EPA directly from microalgae or microbial fermentation processes. However, the current fermentation processes are mainly using sugars, especially glucose, as the starting materials, which limits the yield of the omega-3 products and increases the production cost.

To further improve the microbial production yield of omega-3 EPA from sugars, DuPont has recently developed a new yeast fermentation process, which uses metabolic engineering of Yarrowia lipolytica strains overexpressing desaturase and elongase genes to synthesize omega-3 EPA from glucose under aerobic fermentation conditions. An intermediate strain Y8412 generated by DuPont was deposited in ATCC (strain No. PAT-10026) and became available for further research purpose. Under fed-batch fermentation conditions with glucose as the substrate, the strain Y8412 produced 17% EPA in the yeast biomass.

For biosynthesis of omega-3 fatty acid from glucose, glucose has to be first converted into C16 and C18 fatty acids via glycolysis, TCA cycle, and fatty acid synthesis process. After that, the formed C16-C18 fatty acids are further converted into omega-3 EPA (C20:5) via the newly built desaturation and elongation biosynthesis pathways. However, due to the low efficiency of fatty acid from glucose (typically 1 kg glucose leads to only 0.2-0.25 g C16 to C18 lipids), the yield of omega-3 fatty acid from glucose is still low. Described herein is the use of plant oils or animal fats (with the majority as C16-C18 lipids) directly for biosynthesis of omega-3 production, which will skip the expensive biochemical steps that are required for converting glucose to C16-C18 fatty acid, thus significantly improve the omega-3 production titer and yield. In addition, waste cooking oil (WCO) can be used as the C16-C18 fatty acid source, which further lowers the omega-3 manufacturing cost from the raw materials.

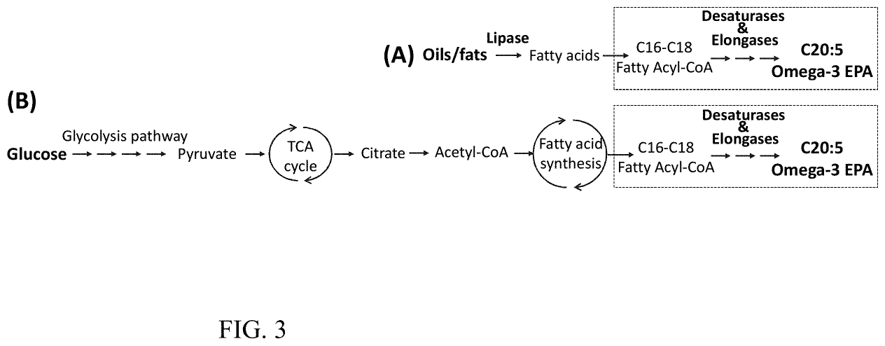

Described herein is a method of using oils/fats as the starting material and directly converting them into omega-3 fatty acids (FIG. 3). This will reduce the number of biochemical reaction steps in the microbial cells, thus significantly improving the biosynthesis efficiency and the overall conversion yield from the starting substrate. In the fed-batch fermentation using both glucose and oils as co-substrates the total omega-3 EPA content in the yeast biomass was improved to 26%, a 50% increase as compared to the run using glucose as the only substrate (see Table 1).

By way of summary, Eicosapentaenoic acid (EPA, C20:5) is chosen as the representative omega-3 product that can be produced from common plant oils and animal fats. A metabolically engineered Y. lipolytica yeast that was originally designed by DuPont for producing omega-3 EPA from glucose was used for oil fermentation experiments. Significantly higher production of omega-3 EPA was achieved by adding oils/fatty acids into the fermentation medium. Adding glycerol into fermentation medium helped convert the molecular format of omega-3 EPA from free fatty acid (FFA) into triacylglyceride (TAG).

A method of directly microbially converting a plant oil, an animal fat, a fatty acid, or a combination thereof, to omega-3 fatty acids comprises growing a microorganism that produces omega-3 fatty acids (e.g., Y. lipolytica strain Y8412 (ATCC #PTA-10026)) in a medium comprising the plant oil, the animal fat, the fatty acid, and optionally glucose, and optionally glycerol, under conditions suitable to produce the omega-3 fatty acids, and optionally isolating the produced omega-3 fatty acids. Optionally, glucose and/or glycerol are added to support cell growth and energy maintenance. Glycerol may be added to the medium to improve the formation of triacylglyceride (or triglyceride) and reduce the content of free fatty acid of the produced omega-3 EPA.

In an aspect, wherein glucose and/or glycerol are added, the weight ratio of total weight of glucose and glycerol to total weight of plant oil, animal fat and fatty acid is between 0:1 to 50:1, preferably between 1:1 to 10:1, more preferably between 5:1 to 10:1.

In another aspect, the plant oil comprises palm oil, soybean oil, corn oil, rapeseed oil, peanut oil, sunflower oil, coconut oil, cotton seed oil, olive oil, or a combination thereof. In yet another aspect, the animal fat comprises beef fat, chicken fat, pork fat, fish fat or oil, or a combination thereof.

In a further aspect, the fatty acid comprises capric acid (C10:0), undecylic acid (C11:0), lauric acid (C12:0), tridecylic acid (C13:0), myristic acid (C14:0), pentadecylic acid (C15:0), palmitic acid (C16:0), palmitoleic acid (C16:1), margaric acid (C17:0), stearic acid (C18:0), oleic acid (C18:1), linoleic acid (C18:2), linolelaidic acid (C18:2), γ-linolenic acid (C18:3), α-linolenic acid (C18:3), nonadecylic acid (C19:0), arachidic acid (C20:0), or a combination thereof.

The plant oil, the animal fat, the fatty acid, or the combination thereof can be sourced from waste cooking oil. The waste cooking oil can be unhydrolyzed or is partially or completely hydrolyzed before adding to the medium.

The medium optionally comprises a lipase.

The invention is further illustrated by the following non-limiting examples.

### EXAMPLES

### Example 1: Construction of Y. lipolytica Strains—VSWE1, 2, 3, 4, and 5

### Materials and Methods

Strain and Plasmids: The wild type Yarrowia lipolytica ATCC20362 was obtained from the American Type Culture Collection (ATCC). The Y. lipolytica strain VSYU1, uracil auxotrophic variant of ATCC20362, was served as the host strain, and all derivative strains used in this study are described in Table 2.

TABLE 2
METABOLICALLY ENGINEERED Y. LIPOLYTICA
STRAINS IN THIS STUDY

| Strain | Genotype | Phenotype | Reference |
|---|---|---|---|
| ATCC20362 | Wild-type | ura⁺ | ATCC |
| VSYU1 | URA3Δ | ura⁻ | This work |
| VSWE1 | VSYU1, MhFAR-WS- | ura⁺ | This work |
| | URA3 | | |
| VSWE2 | VSYU1, MhFAR-WS- | ura⁺ | This work |
| | URA3 | | |
| VSWE3 | VSYU1, MhFAR-WS- | ura⁺ | This work |
| | URA3 | | |
| VSWE4 | VSYU1, MhFAR-WS- | ura⁺ | This work |
| | URA3 | | |
| VSWE5 | VSYU1, MhFAR-WS, | ura⁻ | This work |
| | WS | | |

Cell Culture Medium and Growth Conditions: Y. lipolytica strains ATCC20362 and VSWE1-5 were routinely cultured on plates in a 30° C. stationary incubator or in suspension at 30° C. in a 15-mL conical tubes with rotation of 225 rpm in a New Brunswick G25 shaking incubator. Shake flask cultures (50 mL) were grown in 250-mL Erlenmeyer flasks with air-permeable plugs and were shaken at 30° C. and 225 rpm in a New Brunswick G25 shaking incubator.

The culture medium included Yeast Extract-Peptone-Dextrose (YPD) as the complete medium, Yeast Nitrogen Base (YNB) as the minimal medium for prototroph, and Synthetic Defined medium (SD) for selective growth of auxotroph. YPD medium was formulated using 5 g L⁻¹ yeast extract, 10 g L⁻¹ peptone, 20 g L⁻¹ D-glucose, 1 mg L⁻¹ thiamine HCl, 6 g L⁻¹ KH₂PO₄, and 2 g L⁻¹ Na₂HPO₄. Minimal medium for yeast growth was formulated using 6.7 g L⁻¹ yeast nitrogen base with ammonium sulfate (YNB, Sigma Aldrich), 20 g L⁻¹ D-glucose, 1 mg L⁻¹ thiamine HCl, 6 g L⁻¹ KH₂PO₄, and 2 g L⁻¹ Na₂HPO₄. SD medium was formulated using 6.7 g L⁻¹ yeast nitrogen base with ammonium sulfate (YNB, Sigma Aldrich), 20 g L⁻¹ D-glucose, 1 mg L⁻¹ thiamine HCl, 6 g L⁻¹ KH₂PO₄, 2 g L⁻¹ Na₂HPO₄ and multiple dropout media formulations (e.g. SD-Ura, SD-Leu, SD-Ura-Leu) for auxotrophic selection were generated using the appropriate dropout powder according to the manufacture protocol, as the exact concentration varies with dropout component. Specific components required for growth of auxotrophic strains were added to minimal media to the same concentration as is SD medium. 5-Fluoroorotic Acid (0.5 g L⁻¹ 5-FOA, Zymo Research) and supplementary uracil were both added to SD media plates at 3 g L⁻¹ for URA3 counter-selection. Approximately 20 g L⁻¹ bacteriological agar was added to produce all corresponding media formulations for plating. All culture media for yeast growth were sterilized by autoclaving at 121° C. for half hour.

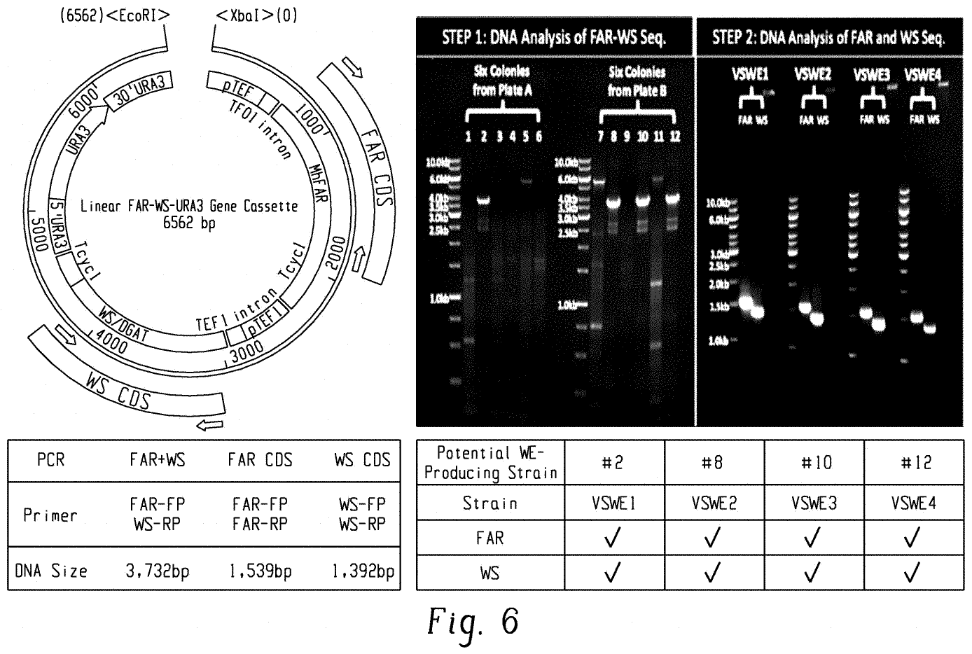

Molecular Biology Protocols: Recombinant wax ester plasmid (pFAR-WS-URA3) was constructed that included two exogenous enzyme genes, fatty acyl-CoA reductase (FAR) and wax ester synthase (WS) for wax ester synthesis, one yeast selection marker (URA3 gene) and the pUC19 plasmid as the backbone (FIG. 5). Both FAR and WS genes were regulated under the control of a TEF1 constitutive promoter and a CYC1 terminator. All the gene sequences required for plasmid assembly were amplified by PCR and ligated via Gibson DNA assembly method. Then, the wax ester plasmid was transformed into a DH5α E. coli strain for plasmid propagation and streaked on the LB/Amp agar plate. The FAR-WS-URA3 gene cassette was linearized from pFAR-WS-URA3 via double digestion for yeast genome editing. The linear FAR-WS-URA3 gene cassette was transformed into the Y. lipolytica VSYU1 strain (the URA3 knockout strain of ATCC20362) and plated on the SD-URA3 plates (FIG. 6). To identify gene transcription, the mRNA was isolated from yeast transformants and detected by reverse-transcription PCR. The molecular weight of the FAR and WS enzymes were confirmed by one-dimensional PAGE that indicated the successful translation from mRNA to protein. Moreover, the function of the enzymes was confirmed by wax ester production, which was determined via the thin layer chromatography (TLC). The growth rates of new yeast strains were then examined in the nitrogen-limited media containing hydrophilic (e.g. glucose) or hydrophobic (e.g. soybean oil and WCO) carbon source in shaking flask and 1-liter fed-batch fermentation scale. Production of fatty alcohols and wax esters was analyzed by gas chromatography (GC).

By using a similar molecular biology tool, a second copy of the WS gene was inserted into the chromosome of the VSWE1 strain, which generated the strain VSWE5 strain with one copy of FAR gene and two copies of WS gene in its chromosome.

Summary: To introduce the wax ester biosynthesis pathway into the Y. lipolytica yeast, the pFAR-WS-URA3 plasmid was first constructed. Then the FAR-WS-URA3 linear cassette (6,562 bp) was extracted and released from the XbaI and EcoRI restriction enzyme double digestions for Y. lipolytica genome editing (FIG. 5). Linearized FAR-WS-URA3 gene cassette was transformed into the Y. lipolytica VSYU1 (the URA3 knockout strain of ATCC20362) via random insertion. Several individual yeast colonies were randomly picked after 72 hours to inoculate the tube-scale culture for plasmid propagation and genomic DNA isolation. The strains with successful transformation were screened by PCR using insert-specific primers. Among 12 picked colonies, four of them were identified to have the FAR and WS genes correctly introduced (FIG. 6). The strains purified from the four colonies were named VSWE1-4, respectively.

### Example 2: Wax Ester Production by the Engineered Y. lipolytica in Shake Flask Experiments

### Methods

Media and growth conditions for shake flask experiments: Wax ester production in Y. lipolytica was performed either in YD, YO or YWCO medium (Table 3). A single transformed colony was selected from a selective plate and used to inoculate a starter culture in minimal media. The starter culture was grown for 24-48 h at 30° C., 250 rpm shaker and was freshly used at a dilution of 1:50 to inoculate expression cultures of YD, YO or YWCO medium for wax ester production. The carbon source of YD media was replaced by 2.48% soybean oil and 2.48% waste cooking oil (canola oil) to make YO and YWCO medium, respectively. Carbon substrate (glucose, oleic acid, soybean oil, or waste cooking oil) was added 2-3 times during the entire culture process (Table 4). Phosphate and carbonate buffer solution containing 6 g L⁻¹ KH₂PO₄, 2 g L⁻¹ K2HPO₄, and 14 g L⁻¹ NaHCO₃ was also added to the expression when necessary for pH adjustment. The flask cultures were typically grown for 5-7 days at 30° C. to reach the maximal wax ester production.

TABLE 3
CULTURE MEDIUM FOR SHAKE FLASK EXPERIMENTS
(50 ML IN EACH 250-L FLASK)

| Component | YD medium | | YO medium | | YWCO medium | |
|---|---|---|---|---|---|---|
| Yeast Extract | 2.5 | g/L | 2.5 | g/L | 2.5 | g/L |
| (NH₄)₂SO₄ | 1 | g/L | 1 | g/L | 1 | g/L |
| Glucose | 40 | g/L | — | | — | |
| Soybean Oil | — | | 24.8 | mL/L | — | |
| Waste cooking oil | — | | — | | 24.8 | mL/L |
| KH₂PO₄ | 6 | g/L | 6 | g/L | 6 | g/L |
| K₂HPO₄ | 2 | g/L | 2 | g/L | 2 | g/L |
| Trace Metal (100X) | 0.5 | mL/L | 0.5 | mL/L | 0.5 | mL/L |
| MgSO₄ | 1 | mL/L | 1 | mL/L | 1 | mL/L |

TABLE 4
FEED SOLUTION FOR SHAKE FLASK EXPERIMENTS (50
ML MEDIUM IN EACH 250-L FLASK, FEEDING THREE
TIMES TO EACH FLASK AT 24 H, 48 H, AND 96 H)

| Component | YD medium | | YO medium | | YWCO medium | |
|---|---|---|---|---|---|---|
| Glucose (500 g/L) | 2 | mL/feed | — | | — | |
| Soybean Oil | — | | 0.625 | mL/feed | — | |
| Waste cooking oil | — | | — | | 0.625 | mL/feed |
| pH Adjusting | 0.35 | mL/feed | 0.35 | mL/feed | 0.35 | mL/feed |
| Buffer (10.5 g/L | | | | | | |
| KH₂PO₄ + 4.15 g/L | | | | | | |
| K₂HPO₄ + 16.7 g/L | | | | | | |
| KHCO₃) | | | | | | |

GC protocols for analysis of wax ester analysis: The extraction of the total wax ester fraction from the engineered Y. lipolytica was performed as the following steps: (1) Cells (500 μL) were harvested and mixed with 500 μL deionized water; (2) The cells were centrifuged 3 min at maximal speed to remove the supernatant; (3) Additional 25 μL of 5 g/L stearyl palmitate (C16:0-Fatty acid/C18:0-Fatty Alcohol, C₃₄H₆₈O₂) (Nu-Check Prep, Inc. USA) as internal wax ester standard was added; (4) Total wax esters were extracted from cells with 500 μL of a mixture of methanol, hexane, deionized water in the ratio 5:1:16 (v/v/v); (5) In each organic solvent adding interval, the cell mixture was homogenized by sonication or vigorous shaking for 30 min; (6) The mixture was centrifuged at 5,000 rpm for 30 sec after agitation; (7) The hexane phase was transferred to chromatography vial; and (8) The extraction was repeated by adding hexane, and the combined supernatant were collected for wax ester quantification via gas chromatography. The separation of individual wax ester was carried out on a GC-2010 gas chromatograph (Shimadzu) equipped with a flame ionization detector. A DB-1HT fused-silica capillary column (15 m×0.25 mm, film thickness of 0.10 μm; Agilent Technologies) was used. The sample (3 μL) was injected at 35° C. Chromatographic separation was initially set at 35° C. for 2 min. The oven was programmed from 35° C. to 240° C. at a rate of 20° C./min and maintained at 240° C. for 6 min, then the temperature was increased from 240° C. to 310° C. at a rate of 20° C./min and maintained for 2 min, and then the temperature was elevated from 310° C. to 360° C. at a rate of 8° C./min. The finial temperature was held for 2 min. Helium was used as a carrier gas at a constant flow rate of 2.0 mL/min. Identification was based in comparison with authentic standard. All data acquisition and processing were performed with the GC-FID solution software (Shimadzu).

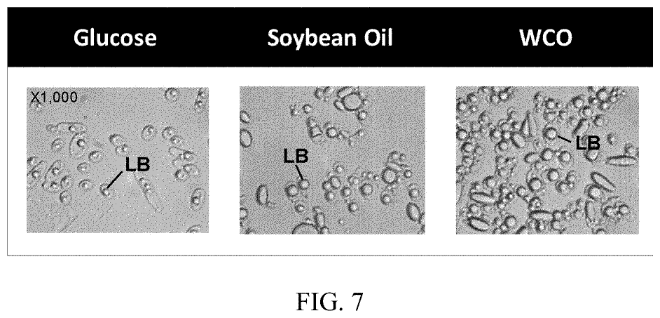

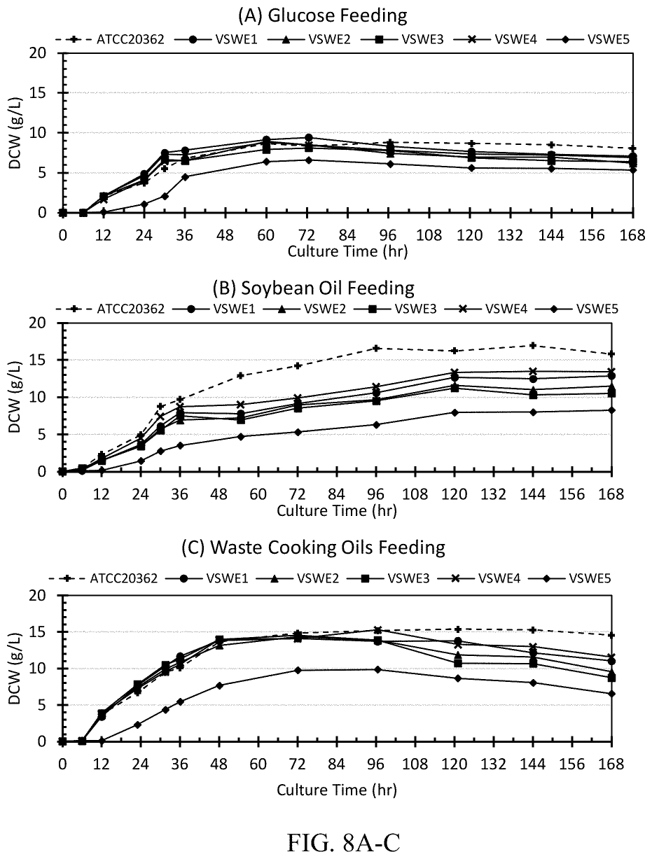

Results: The growth rates of new strains were then examined in the media containing glucose, soybean oil, and WCO (Table 3). From the growth curve, both wild-type and the engineered Y. lipolytica strains exhibited a faster growth rate when the carbon source was changed from the hydrophilic substrate, glucose, to the hydrophobic substrate, soybean oil and waste cooking oil (FIG. 8). About a 50% improvement in dry cell weights (DCW) were observed. Among all five engineered strains, the VSWE1 strain had the fastest growth rate in YD and YWCO medium and produced the highest cell density, which were almost the same as the wild-type Y. lipolytica ATCC20362. In addition, high levels of lipid accumulation within cells were visible under microscopic study (FIG. 7). Larger lipid bodies were formed when soybean oil or waste cooking oil was used as the substrate, while smaller lipid bodies were seen when glucose was the substrate.

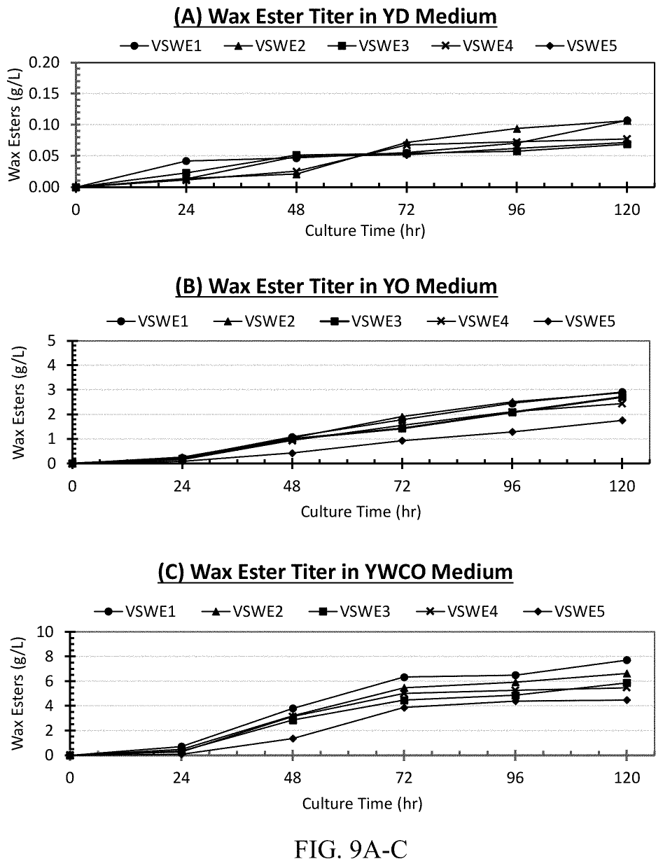

In order to confirm and quantify the desired products in the engineered strains, the GC-MS method was used to analyze the fatty alcohols and wax esters produced in the flask culture. For glucose feeding, the VSWE1 strain has the highest wax ester titer (FIG. 9). The majority of wax esters are formed by esterification of C16:0, C16:1, C18:0, C18:1, and C18:2 fatty alcohols and fatty acids, based on GC and mass spectra analysis. When glucose was used as the only carbon source, all five strains (VSWE1-5) produced about 0.1 g/L wax esters or less after 120 h in flask culture (FIG. 9 A). However, when the carbon source was changed to soybean oil, wax ester production was improved to 2-4 g/L (FIG. 9 B).

When the waste cooking oil collected from a local restaurant (generated from the frying process) was used for the flask fermentation, the wax ester production was almost doubled as compared to the flasks with soybean oil (FIG. 9 C). This result suggested that the engineered Y. lipolytica strains not only grow better and generated 50% more cell mass with fresh soybean oil or waste cooking oil to replace glucose, but also produce 20-70-fold more wax esters (FIG. 9-11).

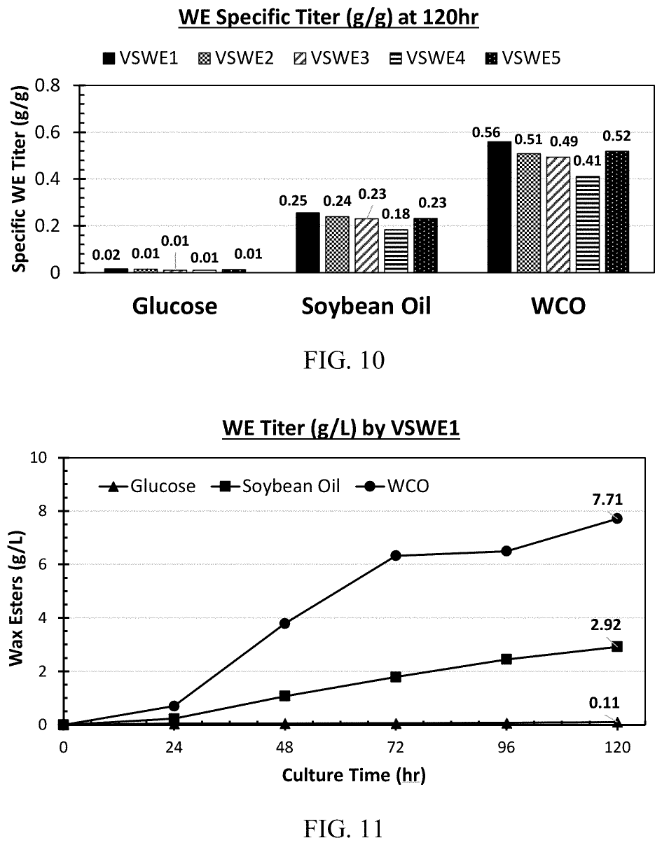

Among all five wax ester strains, VSWE1 grew fastest in oil medium and produced the highest amount of wax ester. This strain produced 7.7 g/L wax esters, this also contributes to 56% of the dry cell weight of VSWE1, which is so far the highest level of wax ester production among all reported in any literature so far. In addition, no improvements in cell growth and wax ester production were seen when a second copy of the WS gene was added in the strain (like VSWE5). All five strains gave very similar specific titer of wax esters in the cell mass (FIG. 10).

### Example 3: Construction of E. coli Strains for Wax Ester Production

Escherichia coli DH5 high efficiency competent cells were used for plasmid construction, preparation, propagation and storage. BL21(DE3) chemically competent E. coli was used for transformation and protein expression. Bacterial host strains and all derivative cloning plasmids used in this study are described in Table 5.

TABLE 5
BACTERIA AND DESIGNED PLASMIDS
FOR CLONING IN THIS STUDY

| | Description | Reference |
|---|---|---|
| E. coli Strain | | |
| DH5α | F⁻ φ80lacZΔM15 Δ(lacZYA- | NEB |
| | argF)U169 recA1 endA1 hsdR17(r_{K}⁻, | |
| | m_{K}⁺) phoA supE44 | |
| | λ⁻ thi-1 gyrA96 relA1 | |
| BL21(DE3) | fhuA2 [lon] ompT gal (λ DE3) [dcm] ΔhsdS | NEB |
| | λ DE3 = λ sBamHIo ΔEcoRI-B | |
| | int::(lacI::PlacUV5::T7 gene1) i21 Δnin5 | |
| Plasmid | | |
| pUC19-FAR- | Amp^{R}, pUC19 derivative that | This work |
| WS-URA3 | expressed FAR from the TEF1 promoter, | |
| | WS from the TEF1 promoter and URA3 | |
| | from the URA3 promoter. | |
| pFAR | Amp^{R}, pET21b(+) derivative | This work |
| | that expressed FAR from the T7 promoter. | |
| pWS | Kan^{R}, pET28a(+) derivative | This work |
| | that expressed WS from the T7 promoter. | |
| pFAR-WS | Amp^{R}, pET21b(+) derivative | This work |
| | that expressed FAR from the T7 promoter | |
| | and WS from the TEF1 promoter. | |
| P(FAR-WS) | Amp^{R}, pET21b(+) derivative | This work |
| | that expressed recombinant FAR and WS | |
| | fusion gene from the T7 promoter. | |

Media and growth conditions: Escherichia coli NEBα were used for transformation and for the amplification of recombinant plasmid DNA was grown at 37° C. in Luria-Bertani (LB) medium supplemented with antibiotic when required. Bacterial strains were cultured on plates in a 37° C. stationary incubator or in suspension at 37° C. in 15-mL conical culture tubes in a New Brunswick G25 shaking incubator operated at 250 rpm. LB medium as the nutrient-rich medium consisted of 5 g L⁻¹ yeast extract, 10 g L⁻¹ tryptone and 10 g L⁻¹ sodium chloride. Approximate 12 g L⁻¹ bacteriological agar was added to solidify LB medium. The addition of 100 μg mL⁻¹ ampicillin (100 mg mL⁻¹ Amp stock concentration) was added for the selection of the bacterial transformants with plasmid carrying the ampicillin resistance gene. All bacterial culture media for growth were sterilized by autoclaving at 121° C. for at least 30 minutes.

The fatty Acyl-CoA reductase (FAR) and wax ester synthase (WS) enzymes were produced in E. coli BL21(DE3) using ZYM-5052 auto-induction medium (1% tryptone, 0.5% yeast extract, 25 mM Na₂HPO₄, 25 mM KH₂PO₄, 50 mM NH₄Cl, 5 mM Na₂SO₄, 2 mM MgSO₄, 0.2× trace elements, 0.5% glycerol, 0.05% glucose and 0.2% α-lactose) supplemented with 100 μg mL⁻¹ ampicillin or 50 μg mL⁻¹ kanamycin. A single transformed colony was selected from a LB plate with antibiotic and used to inoculate a starter culture in LB medium. The starter culture was grown overnight at 37° C. and was freshly used at a dilution of 1:50 to inoculate the expression cultures in ZYM-5052 auto-induction medium. Glycerol was replaced by oleic acid, soybean oil or waste cooking oil for wax ester production in shaking flask experiments. The expression cultures were grown for 30 hr at 37° C. for maximal expression.

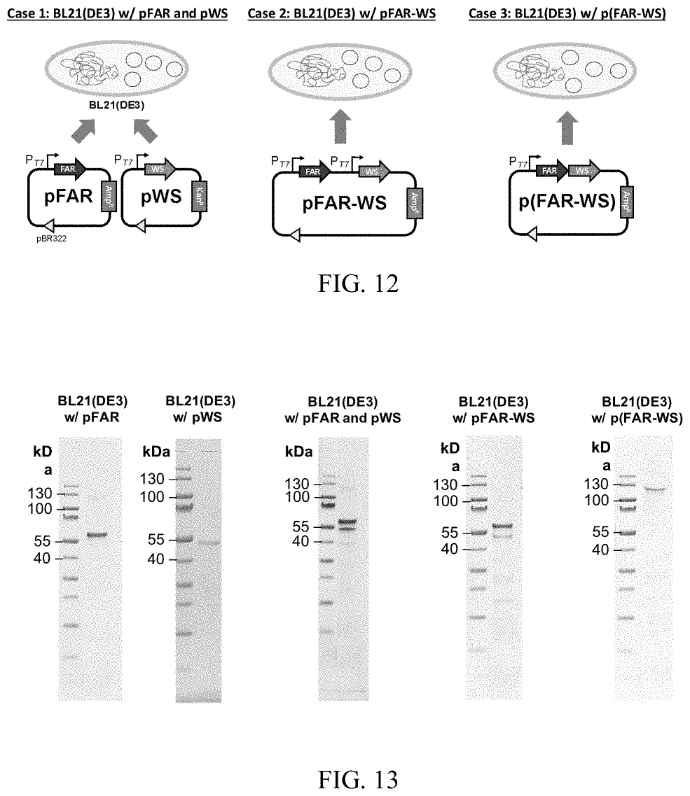

Overview: To establish the E. coli BL21(DE3) bacterial expression system, the pFAR, pWS, pFAR-WS and p(FAR-WS) expression plasmid were designed for the introduction of wax ester biosynthesis pathway into the bacterial cytoplasm for enzyme expression via plasmid transformation (FIG. 12). Engineered bacteria were cultured in the auto-induction medium using different substrates (e.g. glycerol, FFAs, soybean oil and WCO) as carbon source upon the consumption of glucose and alpha-lactose completely. Wax esters extracted from the harvested E. coli cells were confirmed by GC analysis.

High protein expression was achieved to turn on the T7 promoter by adding the inducer IPTG or lactose. To verify the enzyme expression, the polyhistidine-tagged FAR and WS enzymes, extracted from the engineered bacteria, were purified by immobilized metal affinity chromatography. As shown in FIG. 13, the size of synthesized enzymes was confirmed by one-dimensional PAGE analysis with the known molecular weight 57 kDa for FAR, 54 kDa for WS, and 110 kDa for the fused FAR-WS.

### Example 4: Wax Ester Production by Engineered E. coli Strains in Shake Flasks

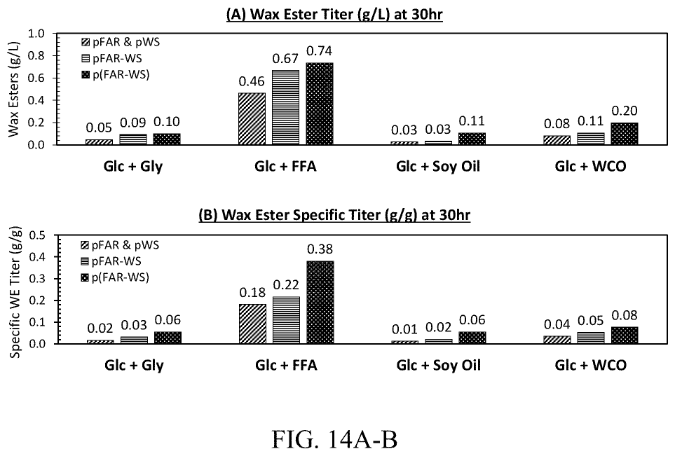

The wax ester production was determined by GC analysis for the newly constructed E. coli strains grown with both hydrophilic (glucose/glycerol) and hydrophobic carbon sources (fatty acids). The E. coli BL21 (DE3) host strains transformed with pFAR and pWS, pFAR-WS or p(FAR-WS) expression vectors was cultured in shake flasks containing ZYM-based medium. Additional carbon sources such as glucose, glycerol, free fatty acids (i.e. oleic acids), lipase-digested soybean oil or WCO was fed as necessary during the flask culturing process. Details of medium and feeding conditions are the same as shown in Table 3 and 4. It was found that wax esters were successfully produced by the engineered E. coli strains. When glucose and glycerol were used as the carbon sources, approximately 38-45 mg/L wax esters were produced. However, only 32-41 mg/L wax esters were produced when soybean oil was used as the sole carbon source. Significantly higher wax ester titers (289-317 mg/L) were achieved when the YO medium containing a mixed carbon source (glucose and oleic acids) was used. Similar wax titers (193-328 mg/L) were also observed by using the YWCO medium containing the mixed glucose and WCO as the substrates. In terms of specific productivity (wax esters/cell density) for the engineered E. coli strains, very similar trend was observed for the three different E. coli strains under different substrate conditions, i.e. the strain BL21(DE3)/p(FAR-WS) with the fused FAR and WS genes produced the highest levels of wax esters (FIG. 14).

### Example 5: Wax Ester Production by E. coli BL21(DE3)/P(FAR-WS) in 1-L Bioreactors

### Methods

The E. coli strain BL21(DE3)/p(FAR-WS) was used for 1-L bioreactor experiments.

Two-stage seed culture: For seed preparation, strains were cultivated on LB agar plates using plate streaking at 37° C. overnight and monoclonal colony was inoculated to 30 mL LB medium to start the first-stage seed culture. The first-stage seed culture was carried out as introduced in the flask culture protocol. When the OD600 reached 3-4, 1 mL of the seed culture was transferred to a 250-mL flask containing 35 mL fresh seed culture medium to grow for 3-4 h at 37° C., 280 rpm in a New Brunswick G25 Shaker Incubator until an OD600 of 1.5-2.5 was reached. The second-stage seed culture was used to inoculate the 1-L fermentor at 5% (v/v).

Fed-Batch Fermentation: The second-stage flask seed culture (30 mL, OD600=1.5-2.5) was transferred to a 1-L fermentor (Biostat® B-DCU, Sartorius, Germany) to initiate the fermentation (t=0 h). The initial fermentation medium was 700 mL and contained citric acid (1.7 g/L), MgSO₄ (0.60 g/L), KH₂PO₄ (14.0 g/L), (NH₄)₂HPO₄ (4.0 g/L), D-Glucose (20.0 g/L), NaCl (0.5 g/L), trace metals I (100×) (10 mL/L), and antifoam (Poly (propylene glycol) monobutyl) (Sigma, America) (1.0 mL/L). The trace metals I (100×) stock solution contained EDTA (840.0 mg/L), CuSO₄.5H₂O (220.0 mg/L), MnCl₂.4H₂O (1500.0 mg/L), CoCl₂.6H₂O (250.0 mg/L), H₃BO₃ (300.0 mg/L), Na₂MoO₄.H₂O (250.0 mg/L), Zn(CH3COO)₂ (1300.0 mg/L), CaCl₂) (1100 mg/L) and ammonium iron(III) citrate (10.0 g/L). The trace metals solution was filter-sterilized through 0.22 μm sterile membrane and stored at 4° C. The dissolved oxygen (measured by pO₂) level of the fermentation experiments was set at 30% of air saturation by cascade controls of agitation speed between 300 and 1200 rpm, gas flow rate between 0.3 lpm and 0.6 lpm, and pure O₂ enrichment (if needed). The temperature was maintained at 37° C. throughout the run. The pH value was maintained at 7.0 throughout the run by feeding ammonium solution (28-30%). Glucose feeding commenced when initial glucose was depleted, which was indicated by rapid increases in pO₂ levels and decreases in agitation speed. Single substrate glucose (600 g/L), dual substrates with both glucose and pure oleic acid or glucose and waste cooking oil (french-fried canola oil obtained from a local restaurant) plus lipase were used for feeding. The feed glucose (600 g/L) containing MgSO₄ (1M) (8 mL/L) and trace metals II (100×) (10 mL/L). The trace metals II (100×) stock solution contained EDTA (1300 mg/L), CuSO₄.5H₂O (370.0 mg/L), MnCl₂.4H₂O (2350.0 mg/L), CoCl₂.6H₂O (400.0 mg/L), H₃BO₃ (500.0 mg/L), Na₂MoO₄.H₂O (400.0 mg/L), Zn(CH3COO)₂ (1600.0 mg/L) and ammonium iron(III) citrate (4.0 g/L). The trace metals solution was filter-sterilized through 0.22 μm sterile membrane and stored at 4° C. Oleic acid or waste cooking oil plus 2% (w/v) MP Biomedicals lipase feeding was commenced after induction. Total of 15 mL of oleic acid or waste cooking oil were to the bioreactor in 3 times. Glucose concentrations were maintained at limited levels (<0.1 g/L) during the fed-batch fermentation by using the pre-set feeding profile. Expression system induction was performed by adding 3 ml, 2.5 mL, and 2.5 mL of IPTG (0.1 in stock solution) at 18 h (the late stage of the exponential growth phase, OD600=50-60), 24 h and 34 h, respectively. A final concentration of IPTG in the medium at 34 h was around 1 mM.

### Results

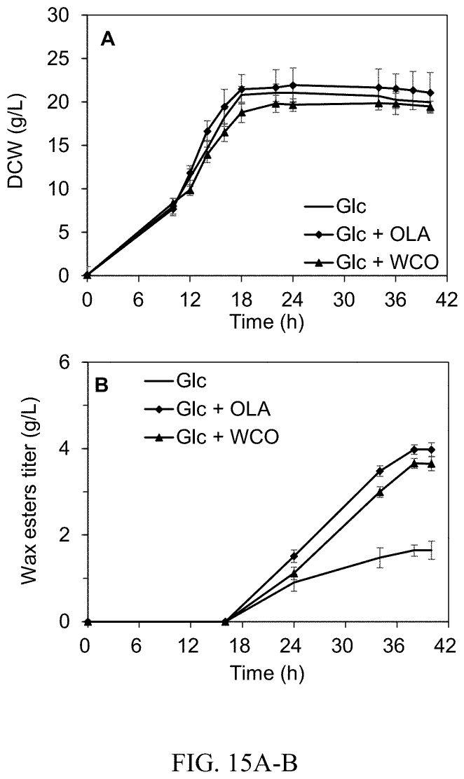

The 1-L fed-batch fermentation of E. coli strain BL21(DE3)/p(FAR-WS) were compared under conditions of using three different carbon sources for wax ester production: (1) glucose, (2) glucose and C18:1 fatty acid, and (3) glucose, waste cooking oil, and lipase. The results are shown in FIG. 15.

When glucose was the only carbon source, a DCW of 20 g/L, a specific titer of 0.08 mg/g DCW and a wax esters titer of 1.7 g/L were obtained at 40 h. However, when oleic acid (C18:1 fatty acid) was co-fed with glucose after about 16 h of the fermentation, a DCW of 21 g/L, a specific titer of 0.2 mg wax esters/g DCW, and a wax esters titer of 4.0 g/L were obtained at 40 h (FIG. 15). When waste cooking oil (WCO) was used to replace the oleic acid (C18:1 FA), lipase was also added to the fermentation medium to decompose the TAG oils into FFAs so that the E. coli cells can directly use the FFAs. As shown in FIG. 15, a DCW of 20 g/L, a specific titer of 0.19 mg/g DCW and a wax esters titer of 3.7 g/L were obtained at 40 h.

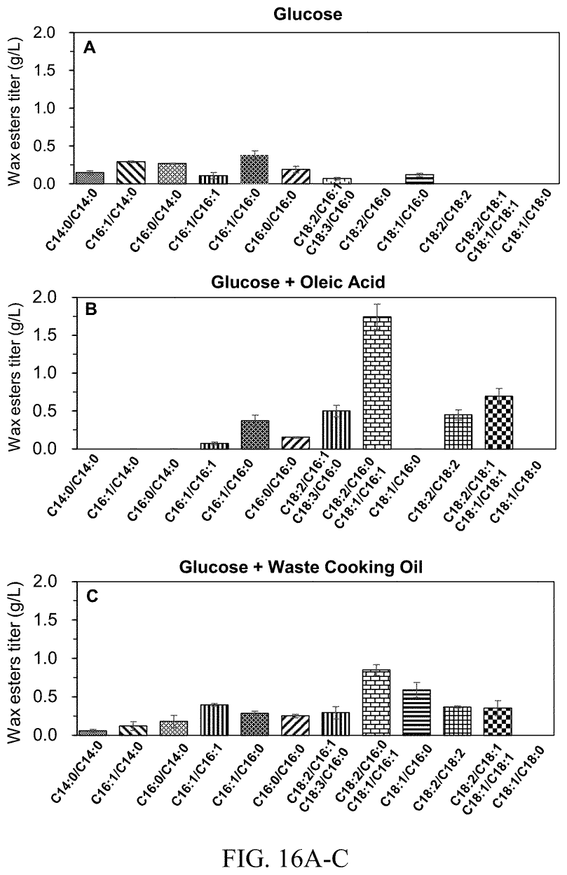

The composition of the produced wax esters is as shown in FIG. 16. Each individual wax ester was determined by comparing the GC results between the fermentation samples and the different authentic samples. Most of the produced wax esters were formed by C14-C16 fatty alcohols and C14-C16 fatty acids when glucose was the only carbon source in the fed-batch fermentation (FIG. 16 A). However, when both glucose and waste cooking oil were used as the carbon sources, the majority of wax esters were formed from C16-C18 fatty alcohols and C16-C18 fatty acids (FIG. 16 C). When C18:1 fatty acid (oleic acid) was used to co-feed with glucose during the fermentation, more wax esters were produced from either C18 fatty alcohols or C18 fatty acid (FIG. 16 B).

Conclusions from Examples 1-5:
1) Both Y. lipolytica yeast and E. coli bacterium were successfully engineered to produce wax esters from glucose, fatty acids, and/or TAG oils or fats.
2) Five engineered Y. lipolytica strains were generated to contain one copy of FAR gene and one copy or two copies of WS gene in its chromosome. In shake flask experiments, the best production strain VSWE1 produced 0.1 g/L wax esters from glucose, 2.9 g/L from soybean oil, and 7.7 g/L from waste cooking oil.
3) Three E. coli strains were also generated to convert free fatty acids into wax esters. In a shake flask experiment, the E. coli strain BL21(DE3) harboring the plasmid p(FAR-WS), which has both FAR and WS genes fused together under a shared T7 promoter, produced 0.7 g/L wax esters from the C18:1 FA and 0.2 g/L wax esters from waste cooking oil.
4) In 1-L fed-batch bioreactors, the engineered E. coli BL21(DE3)/p(FAR-WS) produced about 4 g/L wax esters within 40 h when the C18:1 free fatty acid (oleic acid) or waste cooking oil (WCO) was co-fed with glucose into the fermentation medium.

### Example 6: Microbial Conversion of TAG Oils into Omega-3 Eicosapentaenoic Acid (EPA)

### Methods

Seed culture: The seed vials of Y. lipolytica strain Y8412 stored at −80° C. were thawed for 10 min at room temperature. Inocula were prepared by transferring a 0.5 mL vial solution to a 250 mL shake flask containing 50 mL seed culture medium, which consisted of Bacto™ yeast extract (5 g/L), KH₂PO₄ (6.0 g/L), Na₂HPO₄ (2.0 g/L), D-Glucose (20.0 g/L). The seed cells were grown in shake flasks for 18-24 h at 30° C., 280 rpm in a New Brunswick G25 Shaker Incubator until an OD600 of 2-5 was reached. The seed culture was used to inoculate the 1-L fermentor at 5-7% (v/v).

Fed-batch fermentation: The shake-flask seed culture (50 mL, OD600=2-5) was transferred to a 1-L fermentor (Biostat B-DCU, Sartorius, Germany) to initiate the fermentation (t=0 h). The initial fermentation medium was 0.7 L and contained Bacto™ yeast extract (12.0 g/L), (NH₄)₂SO₄ (9.0 g/L), KH₂PO₄ (6.0 g/L), Na₂HPO₄ (2.0 g/L), D-Glucose (50.0 g/L), MgSO₄.7H₂O (1.2 g/L), thiamin.HCl (1.5 mg/L), trace metals (100×) (2.0 mL/L), and Antifoam 204 (Sigma, 1.0 mL/L). The trace metal (100×) stock solution contained citric acid (15 g/L), CaCl₂).2H₂O (1.5 g/L), FeSO₄.7H₂O (10.0 g/L), ZnSO₄.7H₂O (0.39 g/L), CuSO₄-5H₂O (0.38 g/L), CoCl₂.6H₂O (0.20 g/L), and MnCl₂.4H₂O (0.30 g/L). It was filter-sterilized through 0.22 μm sterile membrane and stored at 4° C. The pO₂ level of the fermentation experiments was set at 30% of air saturation by cascade controls of agitation speed between 500 and 1,400 rpm and pure oxygen enrichment (if needed). The aeration rate was fixed at 0.3 L/min. The temperature was maintained at 30° C. throughout the run. The pH value was controlled at 6.0 during 0-12 h and then gradually increased to 7.0 in 6 hours and maintained at 7.0 in the remainder of the run by feeding KOH (56% w/v). Glucose (600 g/L) and/or glycerol (50% v/v) feeding commenced when its concentration in fermentation medium decreased below 20 g/L. When glucose was used as only substrate, the concentrations were maintained at about 20-30 g/L by adjusting glucose feed rate based on off-line glucose measurements. When glucose was co-fed with waste cooking oil (WCO, french-fried canola oil, provided by a local restaurant), hydrolyzed waste cooking oil (HWCO), or waste cooking oil and lipase (0.02 g/mL oil), WCO or HWCO was added to the medium so that its concentrations were within 5-10 g/L.

For each fermentation with oil co-feeding, a total of 20 mL of WCO/HWCO was fed in 3 pulses at 36 h (10 mL), 48 h (5 mL), and 72 h (10 mL), respectively. The HWCO was obtained by mixing WCO with isometric 0.1 M sodium phosphate buffer (pH=7.2), which contains 1% MP Biomedicals lipase (w/v), at 37° C. for 48 h. After the reaction, the lipid layer was collected by centrifugation and around 63% of oil was converted to free fatty acids.

GC analysis of total fatty acids (TFAs) and EPA production: Fatty acids (FAs) synthesized by Y. lipolytica were quantified using gas chromatography coupled to a flame ionization detector (GC-FID).

Prior to GC analysis, the intracellular and extracellular fatty acids (triglycerides (TAGs) and free fatty acids (FFAs)) were converted into their fatty acid methyl esters (FAMEs) by base-catalyzed or acid-catalyzed reaction. The base-catalyzed transesterification method was used to analyze the total TAGs. The acid-catalyzed transesterification and esterification method was used to analyze both TAGs and FFAs.

### Base-Catalyzed Transesterification

Base-catalyzed transesterification using 1% sodium methoxide solution in methanol. TAGs are transesterified in anhydrous methanol in the presence of a basic catalyst. Free fatty acids are not normally esterified.

Cells from 0.1 ml of fermentation broth were collected by centrifugation at 7500 rpm for 3 min and the supernatant was transferred to a new micro-centrifuge tube. The pellets were washed with distilled water for 3 times. The supernatant was concentrated on a Savant SPD1010 SpeedVac™ concentrator (Thermo Scientific). A total of 100 μL internal standard containing 5 mg/mL methyl pentadecanoate (Nu-Check Prep, Inc. USA) and 5 mg/mL glyceryl triheptadecanoate (Nu-Check Prep, Inc. USA) dissolved in heptane was added to each sample as an internal standard. Methyl pentadecanoate was used for volume loss correction during sample preparation and glyceryl triheptadecanoate was used for transesterification efficiency correction. A total of 500 μL of 1% sodium methoxide solution (in methanol) was then added each pellet and concentrated supernatant sample for transesterification into FAMEs. The samples were placed in a shaker at room temperature for 45 min. After the reaction, 200 μl of 1.0 M sodium chloride and 1000 μl of heptane were added to each sample, which was then vortexed for 30 min at 1,200 rpm (VWR vortex mixer) for FAME extraction. Two layers were formed after the sample was centrifuged at 6,000×g for 30 s. The top heptane layer containing the FAMEs was collected and used for GC analysis.

### Acid-Catalyzed Transesterification and Esterification

Acid-catalyzed reaction using 2.5% sulfuric acid in methanol. In this reaction, both TAGs and FFAs were converted to FAMEs by transesterification and esterification, respectively.

Cells from 0.1 ml of broth were collected by centrifugation in a 15-mL centrifuge tube. The supernatant was transferred to a new 15-mL tube. Pellets were washed with distilled water for 3 times and the supernatant was removed. A total of 100 μL internal standard containing 5 mg/mL methyl pentadecanoate (Nu-Check Prep, Inc. USA) and 5 mg/mL glyceryl triheptadecanoate (Nu-Check Prep, Inc. USA) dissolved in heptane was added to each sample as an internal standard. Then 1000 μL of 2.5% sulfuric acid in methanol were added to the cell pellets and to the supernatant in a new tube for acid-catalyzed transesterification and esterification reaction. The samples were placed in a water bath at 80° C. (vortex interval) for 60 min. After cooling down, 100 μl of 1M sodium chloride and 1000 μl of heptane were added to each sample. All samples were vortexed and centrifuged to further separate the top heptane layer containing the FAMEs.

GC analysis of FAMEs was performed with a Shimadzu GC2010 Plus GC with a flame ionization detector (FID). The samples were injected into the GC/FID system equipped with a Shimadzu Rxi-5 ms column (15 m). The GC was programmed with the following inlet operating parameters: high purity helium carrier gas set at a constant flow pressure of 150 kPa, inlet temperature set at 265° C., and split injection mode with split ratio of 150. The detector temperature was set at 265° C., with an air flow rate of 400 mL/min, a hydrogen gas flow rate of 40 mL/min, and a makeup gas flow rate of 30 mL/min. The GC oven was programmed with the following temperature regime: start at 35° C., hold for 1.5 min, ramp up to 175° C. at 20° C./min and hold for 3 min, ramp to 195° C. at 15° C./min, and ramp to 265° C. at 20° C./min and then hold 265° C. for 2 min. Quantification of fatty acids was achieved through standard curves measuring commercial FAME standards purchased from Nu-Check Prep, Inc. (MN, USA). Identification of the FAMEs was achieved using a Shimadzu GC2010 Plus GC equipped with a mass spectrometer (MS).

### Results

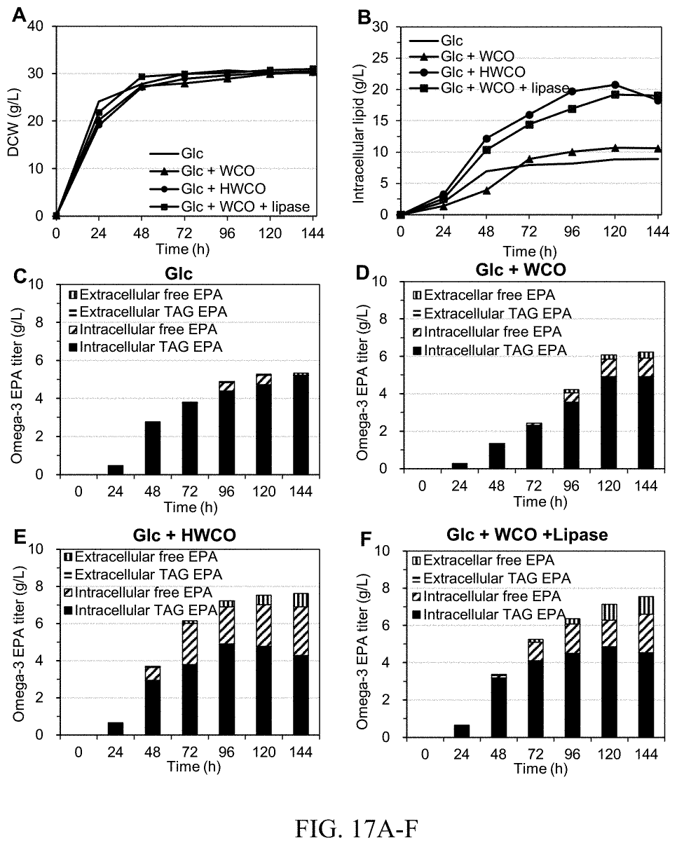

Omega-3 EPA production from glucose by Y. lipolytica Y8412 in 1-L bioreactors: Cell growth and EPA production of the Y. lipolytica was first studied as a control in a 1-L bioreactor by using glucose as the only carbon source, as described in the fed-batch fermentation protocols. As shown in FIG. 17 (A-C) and Table 6, a DCW of 30.9 g/L, a total EPA titer of 5.3 g/L and a final TFAs titer of 8.9 g/L were obtained at 144 h. In addition, the EPA was mainly trieicosapentaenoic (EPA in the format of triacylglyceride). The composition of the total fatty acids (TFAs) is shown in Table 6.

Improved omega-3 EPA production from WCO by Y. lipolytica Y8412: Since the β-oxidation pathway in strain Y8412 was inactivated and the strain cannot directly utilize fatty acids, glucose was still used as the only carbon source during cell growth phase (0-36 h). In production phase (t>36 h), a total of 20 mL WCO or hydrolyzed WCO (HWCO) was fed in three pulses into the fermentation medium (700 mL), with 10 mL at 36 h, 5 mL at 48 h, and 5 mL at 72 h, respectively.

TABLE 6
TOTAL LIPID CONTENT AND COMPOSITION IN YARROWIA STRAIN Y8412
WITH DIFFERENT SUBSTRATES IN FED-BATCH FERMENTATION AT 144 H.

| | | | % TFAs | | | | | | |
|---|---|---|---|---|---|---|---|---|---|
| | DCW | Lipid | | | | | | EPA | EPA |
| Substrates | (g/L) | (% DCW) | C16:0 | C16:1 | C18:0 | C18:1 | C18:2 | C20:5 | (% DCW) |
| Glc | 30.9 | 28.7% | 4.0% | 0.4% | 4.1% | 6.5% | 17.2% | 58.9% | 17.2% |
| Glc + WCO | 30.3 | 33.8% | 2.6% | 0.8% | 3.7% | 9.7% | 20.3% | 57.8% | 20.1% |
| Glc + HWCO | 30.1 | 52.1% | 1.1% | 0.2% | 3.3% | 17.8% | 26.1% | 42.1% | 25.9% |
| Glc + WCO + | 31.0 | 52.8% | 4.7% | 0.5% | 4.3% | 18.0% | 23.3% | 39.1% | 24.2% |
| lipase | | | | | | | | | |

*Glc: glucose, WCO: waste cooking oil, HWCO: hydrolyzed waste cooking oil, Gly: glycerol.

As shown in FIG. 17 (A, B, D) and Table 6, a DCW of 30.3 g/L, a total EPA titer of 6.1 g/L and a TFA titer of 10.5 g/L were obtained at 144 h when three pulses of WCO was fed at 36 h (10 mL), 48 h (5 mL), and 72 h (5 mL), respectively. Compared with the fermentation with glucose feeding only, co-feeding with WCO from 36 h improved the overall EPA and TFA production by 15% and 18%, respectively.

### Improving Omega-3 EPA Production by Using Hydrolyzed WCO (HWCO)

In order to further improve the omega-3 EPA production, hydrolyzed WCO (HWCO) instead of WCO was co-fed with glucose. A total of 20 mL HWCO was fed in three pulses at 36 h (10 mL), 48 h (5 mL), and 72 h (5 mL), respectively. As shown in FIG. 17 (A, B, E) and Table 6, a DCW of 30.1 g/L, a total EPA titer of 7.8 g/L and a final TFAs titer of 18.3 g/L were obtained at 144 h. Compared with the fermentation with glucose feeding only, the overall EPA and TFAs production were improved by 49% and 106%, respectively.

Improving Omega-3 EPA Production from WCO by Adding Lipase to Medium

Since HWCO requires pretreating WCO with lipase, which brings in extra production cost and increases the process complexity, in the new fermentation experiment the non-pretreated WCO was fed to the bioreactor, but additional lipase was added into the medium at a ratio of 0.02 g lipase/mL WCO to help decompose the WCO into more FFAs in the fermentation medium to facilitate the TAG oil utilization. As shown in FIG. 17 (A, B, F) and Table 6, a DCW of 31.0 g/L, a total EPA titer of 7.5 g/L, and a final TFA titer of 19.0 g/L were obtained at 144 h. The total EPA titer and TFA titer achieved were very similar to the previous fermentation run with co-feeding of HWCO, but the process of co-feeding WCO and lipase without pretreating WCO with lipase is much simpler to apply in a larger scale fermentation process.

Though co-feeding HWCO or WCO plus lipase significantly improved production of EPA and TFAs, the fraction of free fatty acid of EPA in total EPA (30-45%) were much higher than that from the fermentation using glucose feeding only (2.6%). In addition, a high content of intracellular free fatty acid may cause toxicity to the yeast cells, as a result about 4%-15% of EPA (in the format of FFA) was secreted to fermentation medium. Our observation of increased levels of FFA secretion in the late stationary phase is also consist with what was observed in the art. Because TAG is not as toxic as free fatty acid to cells and it cannot be secreted (no extracellular TAG EPA was detected and no TAG transported has been reported), converting EPA free fatty acid to TAG would be beneficial for EPA production. Therefore, converting EPA's format from FFA to TAG will also keep the TAG EPA inside the cells, which will avoid the extra effort to recover the extracellular EPA FFA in the medium. Since TAG formation requires glycerol as the backbone to esterify with FFA, in Example 7 we explored the use of glycerol to partially or completely replace glucose to improve the TAG formation of the produced EPA.

### Example 7: Feeding Glycerol to Help Convert FFA EPA into TAG EPA

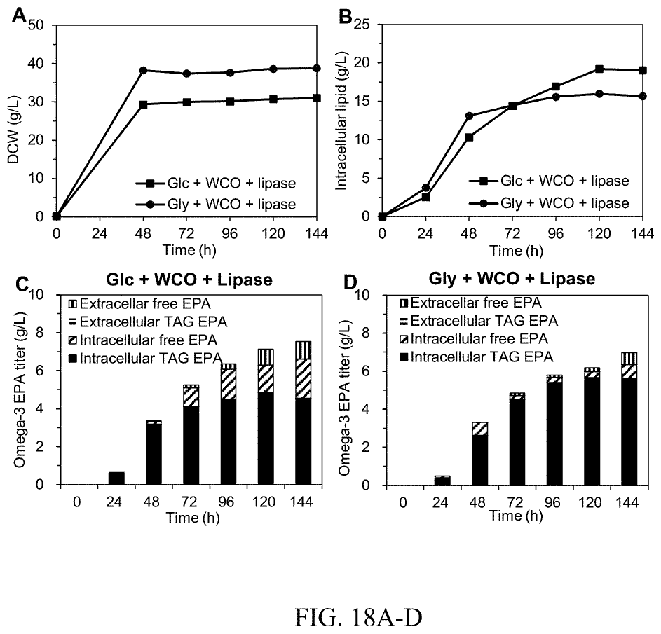

In the new fermentation experiment glycerol was used to completely replace glucose as the main carbon source to support cell growth and maintenance. The initial glycerol concentration in the medium was 20 g/L. After the initial glycerol was consumed, which was indicated by a quick increase in pO₂, a quick decrease in agitation speed, and a slow increase in pH value. Glycerol was fed to maintain its residual concentrations at around 10 g/L. Co-feeding WCO and lipase started from 36 h. A total of 20 mL WCO was fed in three pulses at 36 h (10 mL), 48 h (5 mL) and 72 h (5 mL), respectively. Lipase was also fed together with WCO at a ratio of 0.02 g lipase/mL WCO. As shown in FIG. 18 (A-D), a DCW of 37.0 g/L, a total EPA titer of 6.8 g/L, and a TFAs titer of 17.4 g/L were obtained at 144 h. Although, the total EPA and TFA production slightly decreased as compared to the fermentation with glucose and WCO plus lipase, the percentage of TAG EPA in total EPA was improved from 60.2% to 83.1%. This suggests that using more glycerol in the medium still showed a great potential to convert more FFA to TAG for the produced omega-3 EPA.

### CONCLUSIONS

Conclusions include the following:
(1) Co-feeding waste cook oil (WCO) with glucose during the fed-batch omega-3 fermentation significantly improved the accumulation of intracellular lipids and the total omega-3 EPA production.
(2) About 20%-40% of the produced omega-3 EPA was produced in the format of intracellular free fatty acids when WCO was used as substrates. In addition, 4%-15% of the produced EPA was released to the medium as extracellular free fatty acid.
(3) When the WCO was co-fed with glucose during 36-72 h, about 5.8 g/L intracellular EPA and 0.32 g/L extracellular EPA was produced. The total EPA production was improved by 18% as compared to the fermentation with glucose feeding only, where 5.2 g/L EPA was produced.
(4) When the WCO and lipase were co-fed with glucose during 36-72 h, about 6.6 g/L intracellular EPA and 0.93 g/L extracellular EPA was produced. The total EPA production was improved by 45% as compared to the fermentation with glucose feeding only.
(5) When the WCO was pretreated with lipase and then the hydrolyzed WCO (HWCO) was co-fed with glucose during 36-72 h, about 7.1 g/L intracellular EPA and 0.7 g/L extracellular EPA was produced. The total EPA production was improved by 49% as compared to the fermentation with glucose feeding only.
(6) Adding more glycerol to the fed-batch fermentation medium showed a great potential to minimize the portion of free fatty acid format in the produced omega-3 EPA.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of directly microbially converting a plant oil, an animal fat, free fatty acid, or a combination thereof to wax esters, comprising
growing a bacterial strain in a medium comprising the plant oil, the animal fat, or a combination thereof, under conditions suitable to produce the wax esters, wherein the bacterial strain is engineered to express a FAR gene encoding fatty acid alcohol reductase and a WS gene encoding a wax ester synthase,
or
growing a yeast strain in a medium comprising the plant oil, the animal fat, the free fatty acid, or a combination thereof, under conditions suitable to produce the wax esters, wherein the yeast strain is engineered to express a FAR gene encoding fatty acid alcohol reductase and a WS gene encoding a wax ester synthase, and
optionally isolating the produced wax esters.

2. The method of claim 1, wherein the microbe is a bacterial or yeast strain and the medium is glucose-free.

3. The method of claim 1, wherein the plant oil comprises palm oil, soybean oil, corn oil, rapeseed oil, peanut oil, sunflower oil, coconut oil, cotton seed oil, olive oil, or a combination thereof, and wherein the animal fat comprises beef fat, chicken fat, pork fat, fish fat or oil, or a combination thereof.

4. The method of claim 1, wherein the fatty acid comprises capric acid (C10:0), undecylic acid (C11:0), lauric acid (C12:0), tridecylic acid (C13:0), myristic acid (C14:0), pentadecylic acid (C15:0), palmitic acid (C16:0), palmitoleic acid (C16:1), margaric acid (C17:0), stearic acid (C18:0), oleic acid (C18:1), linoleic acid (C18:2), linolelaidic acid (C18:2), γ-linolenic acid (C18:3), α-linolenic acid (C18:3), nonadecylic acid (C19:0), arachidic acid (C20:0), or a combination thereof.

5. The method of claim 1, wherein the medium further comprises a lipase.

6. The method of claim 5, wherein the lipase is produced by the yeast or bacterial strain, provided in the medium, fed during the growth process, or a combination thereof.

7. The method of claim 1, wherein the FAR gene comprises MhFAR from M. hydrocarbonoclasticus, AmFAR from Apis mellifera, HsFAR from Homo sapiens brain tissue, AtFAR5 from Arabidopsis thaliana, or MmFAR from Mus musculus.

8. The method of claim 1, wherein the WS gene is from the Jojoba Simmondsia chinensis or from a microorganism selected from A. calcoaceticus, A. baylyi, M hydrocarbonoclasticus, R. opacus, and P. arcticus.

9. The method of claim 1, wherein the yeast strain is an oleaginous yeast selected from Y. lipolytica, Rhodosporidium toruloides, Lipomyces starkey, Rhodotorula glutinis, Trichosporon fermentans, and Cryptococcus curvatus, or wherein the bacteria is E. coli, Bacillus subtilis, Streptococcus pneumonia, or another bacterial strain that can metabolize a fatty acid substrate.

10. The method of claim 9, wherein the bacteria E. coli uses a T7 RNA polymerase system for expression of the FAR and WS genes.

11. A Yarrowia lipolytica ATCC20362 strain engineered to express a FAR gene encoding fatty acid alcohol reductase and a WS gene encoding a wax ester synthase.

12. A method of directly microbially converting a plant oil, an animal fat, a fatty acid, or a combination thereof, to omega-3 fatty acids, comprising
growing a microorganism that produces omega-3 fatty acids in a medium comprising
the plant oil, the animal fat, the fatty acid, and
optionally glucose, and
optionally glycerol,
under conditions suitable to produce the omega-3 fatty acids, and
optionally isolating the produced omega-3 fatty acids.

13. The method of claim 12, wherein glucose and/or glycerol are added, and the weight ratio of total weight of glucose and glycerol to total weight of plant oil, animal fat and fatty acid is between 0:1 to 50:1, preferably between 1:1 to 10:1, more preferably between 5:1 to 10:1.

14. The method of claim 12, wherein the plant oil comprises palm oil, soybean oil, corn oil, rapeseed oil, peanut oil, sunflower oil, coconut oil, cotton seed oil, olive oil, or a combination thereof, and wherein the animal fat comprises beef fat, chicken fat, pork fat, fish fat or oil, or a combination thereof.

15. The method of claim 12, wherein the fatty acid comprises capric acid (C10:0), undecylic acid (C11:0), lauric acid (C12:0), tridecylic acid (C13:0), myristic acid (C14:0), pentadecylic acid (C15:0), palmitic acid (C16:0), palmitoleic acid (C16:1), margaric acid (C17:0), stearic acid (C18:0), oleic acid (C18:1), linoleic acid (C18:2), linolelaidic acid (C18:2), γ-linolenic acid (C18:3), α-linolenic acid (C18:3), nonadecylic acid (C19:0), arachidic acid (C20:0), or a combination thereof.

16. The method of claim 12, wherein the plant oil, the animal fat, the fatty acid, or the combination thereof is sourced from unused products, used or waste products of a plant oil, animal fat, and fatty acid, which include but not limited to waste cooking oil and rendered animal fats.

17. The method of claim 16, wherein the plant oil or animal fat is unhydrolyzed or is partially or completely hydrolyzed before adding to the medium.

18. The method of claim 12, wherein the medium further comprises a lipase.

19. The method of claim 18, wherein the lipase is produced by the yeast or bacterial strain, provided in the medium, fed during the growth process, or a combination thereof.


SEQUENCE LISTING (10 sequences)

SEQ ID NO: 1 - protein, 513 aa, Marinobacter hydrocarbonoclasticus
MAIQQVHHADTSSSKVLGQLRGKRVLITGTTGFLGKVVLERLIRAVPDIGAIYLLIRGNKRHPDARSRFLEEIATSSVFDRLREADSEGFDAFLEERIHCVTGEVTEAGFGIGQEDYRKLATELDAVINSAASVNFREELDKALAINTLCLRNIAGMVDLNPKLAVLQVSTCYVNGMNSGQVTESVIKPAGEAVPRSPDGFYEIEELVRLLQDKIEDVQARYSGKVLERKLVDLGIREANRYGWSDTYTFTKWLGEQLLMKALNGRTLTILRPSIIESALEEPAPGWIEGVKVADAIILAYAREKVTLFPGKRSGIIDVIPVDLVANSIILSLAEALGEPGRRRIYQCCSGGGNPISLGEFIDHLMAESKANYAAYDHLFYRQPSKPFLAVNRALFDLVISGVRLPLSLTDRVLKLLGNSRDLKMLRNLDTTQSLATIFGFYTAPDYIFRNDELMALANRMGEVDKGLFPVDARLIDWELYLRKIHLAGLNRYALKERKVYSLKTARQRKKAA

SEQ ID NO: 2 - protein, 516 aa, Apis mellifera
MSTISDNQCTSVRDFYKDRSIFITGGTGFMGKVLVEKLLRSCPGIKNIYILMRPKKSQDIQQRLQKLLDVPLFDKLRRDTPDELLKIIPIAGDVTEHELGISEADQNVIIRDVSIVFHSAATVKFDEPLKRSVHINMIGTKQLLNLCHRMHNLEALIHVSTAYCNCDRYDVAEEIYPVSAEPEEIMALTKLMDSQMIDNITPTLIGNRPNTYTFTKALTERMLQSECGHLPIAIVRPSIVLSSFREPVSGWVDNLNGPTGIVAAAGKGFFRSMLCQKNMVADLVPVDIVINLMICTAWRTATNRTKTIPIYHCCTGQQNPITWQQFVELILKYNRMHPPNDTIWWPDGKCHTFAIVNNVCKLFQHLLPAHILDFIFRLRGKPAIMVGLHEKIDKAVKCLEYFTMQQWNFRDDNVRQLSGELSPEDRQIFMFDVKQIDWPSYLEQYILGIRQFIIKDSPETLPAARSHIKKLYWIQKVVEFGMLLVVLRFLLLRIPMAQSACFTLLSAILRMCRMIV

SEQ ID NO: 3 - protein, 515 aa, Homo sapiens
MVSIPEYYEGKNVLLTGATGFLGKVLLEKLLRSCPKVNSVYVLVRQKAGQTPQERVEEVLSGKLFDRLRDENPDFREKIIAINSELTQPKLALSEEDKEVIIDSTNIIFHCAATVRFNENLRDAVQLNVIATRQLILLAQQMKNLEVFMHVSTAYAYCNRKHIDEVVYPPPVDPKKLIDSLEWMDDGLVNDITPKLIGDRPNTYIYTKALAEYVVQQEGAKLNVAIVRPSIVGASWKEPFPGWIDNFNGPSGLFIAAGKGILRTIRASNNALADLVPVDVVVNMSLAAAWYSGVNRPRNIMVYNCTTGSTNPFHWGEVEYHVISTFKRNPLEQAFRRPNVNLTSNHLLYHYWIAVSHKAPAFLYDIYLRMTGRSPRMMKTITRLHKAMVFLEYFTSNSWVWNTENVNMLMNQLNPEDKKTFNIDVRQLHWAEYIENYCLGTKKYVLNEEMSGLPAARKHLNKLRNIRYGFNTILVILIWRIFIARSQMARNIWYFVVSLCYKFLSYFRASSTMRY

SEQ ID NO: 4 - protein, 491 aa, Arabidopsis thaliana
MESNCVQFLGNKTILITGAPGFLAKVLVEKILRLQPNVKKIYLLLRAPDEKSAMQRLRSEVMEIDLFKVLRNNLGEDNLNALMREKIVPVPGDISIDNLGLKDTDLIQRMWSEIDIIINIAATTNFDERYDIGLGINTFGALNVLNFAKKCVKGQLLLHVSTAYISGEQPGLLLEKPFKMGETLSGDRELDINIEHDLMKQKLKELQDCSDEEISQTMKDFGMARAKLHGWPNTYVFTKAMGEMLMGKYRENLPLVIIRPTMITSTIAEPFPGWIEGLKTLDSVIVAYGKGRLKCFLADSNSVFDLIPADMVVNAMVAAATAHSGDTGIQAIYHVGSSCKNPVTFGQLHDFTARYFAKRPLIGRNGSPIIVVKGTILSTMAQFSLYMTLRYKLPLQILRLINIVYPWSHGDNYSDLSRKIKLAMRLVELYQPYLLFKGIFDDLNTERLRMKRKENIKELDGSFEFDPKSIDWDNYITNTHIPGLITHVLKQ

SEQ ID NO: 5 - protein, 515 aa, Mus musculus
MVSIPEYYEGKNILLTGATGFLGKVLLEKLLRSCPRVNSVYVLVRQKAGQTPQERVEEILSSKLFDRLRDENPDFREKIIAINSELTQPKLALSEEDKEIIIDSTNVIFHCAATVRFNENLRDAVQLNVIATRQLILLAQQMKNLEVFMHVSTAYAYCNRKHIDEVVYPPPVDPKKLIDSLEWMDDGLVNDITPKLIGDRPNTYIYTKALAEYVVQQEGAKLNVAIVRPSIVGASWKEPFPGWIDNFNGPSGLFIAAGKGILRTMRASNNALADLVPVDVVVNTSLAAAWYSGVNRPRNIMVYNCTTGSTNPFHWGEVEYHVISTFKRNPLEQAFRRPNVNLTSNHLLYHYWIAVSHKAPAFLYDIYLRMTGRSPRMMKTITRLHKAMVFLEYFTSNSWVWNTDNVNMLMNQLNPEDKKTFNIDVRQLHWAEYIENYCMGTKKYVLNEEMSGLPAARKHLNKLRNIRYGFNTILVILIWRIFIARSQMARNIWYFVVSLCYKFLSYFRASSTMRY

SEQ ID NO: 6 - protein, 352 aa, Simmondsia chinensis
MEVEKELKTFSEVWISAIAAACYCRFVPAVAPHGGALRLLLLLPVVLLFIFLPLRLSSFHLGGPTALYLVWLANFKLLLFAFHLGPLSNPSLSLLHFISTTLLPIKFRDDPSNDHEKNKRTLSFEWRKVVLFVAKLVFFAGILKIYEFRKDLPHFVISVLYCFHFYLGTEITLAASAVIARATLGLDLYPQFNEPYLATSLQDFWGRRWNLMVSDILGLTTYQPVRRVLSRWVRLRWEVAGAMLVAFTVSGLMHEVFFFYLTRARPSWEVTGFFVLHGVCTAVEMVVKKAVSGKVRLRREVSGALTVGFVMVTGGWLFLPQLVRHGVDLKTIDEYPVMFNYTQKKLMGLLGW

SEQ ID NO: 7 - protein, 458 aa, Acinetobacter baylyi
MRPLHPIDFIFLSLEKRQQPMHVGGLFLFQIPDNAPDTFIQDLVNDIRISKSIPVPPFNNKLNGLFWDEDEEFDLDHHFRHIALPHPGRIRELLIYISQEHSTLLDRAKPLWTCNIIEGIEGNRFAMYFKIHHAMVDGVAGMRLIEKSLSHDVTEKSIVPPWCVEGKRAKRLREPKTGKIKKIMSGIKSQLQATPTVIQELSQTVFKDIGRNPDHVSSFQAPCSILNQRVSSSRRFAAQSFDLDRFRNIAKSLNVTINDVVLAVCSGALRAYLMSHNSLPSKPLIAMVPASIRNDDSDVSNRITMILANLATHKDDPLQRLEIIRRSVQNSKQRFKRMTSDQILNYSAVVYGPAGLNIISGMMPKRQAFNLVISNVPGPREPLYWNGAKLDALYPASIVLDGQALNITMTSYLDKLEVGLIACRNALPRMQNLLTHLEEEIQLFEGVIAKQEDIKTAN

SEQ ID NO: 8 - protein, 455 aa, Marinobacter hydrocarbonoclasticus
MTPLNPTDQLFLWLEKRQQPMHVGGLQLFSFPEGAPDDYVAQLADQLRQKTEVTAPFNQRLSYRLGQPVWVEDEHLDLEHHFRFEALPTPGRIRELLSFVSAEHSHLMDRERPMWEVHLIEGLKDRQFALYTKVHHSLVDGVSAMRMATRMLSENPDEHGMPPIWDLPCLSRDRGESDGHSLWRSVTHLLGLSDRQLGTIPTVAKELLKTINQARKDPAYDSIFHAPRCMLNQKITGSRRFAAQSWCLKRIRAVCEAYGTTVNDVVTAMCAAALRTYLMNQDALPEKPLVAFVPVSLRRDDSSGGNQVGVILASLHTDVQDAGERLLKIHHGMEEAKQRYRHMSPEEIVNYTALTLAPAAFHLLTGLAPKWQTFNVVISNVPGPSRPLYWNGAKLEGMYPVSIDMDRLALNMTLTSYNDQVEFGLIGCRRTLPSLQRMLDYLEQGLAELELNAGL

SEQ ID NO: 9 - protein, 460 aa, Rhodococcus opacus
VGDESHRGDVRAVRDAVASHARGCARTVRTAARVGARSCPVHVRGVDLARGRVGHVPPARRTPAPRRQLPLVVVRRPHRSRLPRPAHRGPGPGPHGGPAVAGVPDARHAARSAAPDVGDARHRGARRRTDGRVLENSPVADGRTGRAAASPSRPVDGPRRPRLPRTVDARGRRHVAAGIRAAGGRGAGRVRAATSIVGVLPALAKVAYDGVREQHLTLPLQSPPTMLNVPVGRARKLAARSWPIRRLVSVATAASTTINAVVLAMCSGALRRYLLEQYALPDAPLTAMLPVPLDLGGTMIGPRGRDHGVGAMVVGLATDEADPAARLARISESVEHTNRVFGALSHTQFQLMSALAISPILLEPVRRFVDDTPPPFNVMISYMPGPSRPRYWNGARLDAVYPAPTVLGGQALSITLTSRSGQLDVGVVGDRHAVPHLQRIITHLETSLSDLEHAVAASGT

SEQ ID NO: 10 - protein, 475 aa, Psychrobacter arcticus
MRLLTAVDQLFLLLESRKHPMHVGGLFLFELPENADISFVHQLVKQMQDSDVPPTFPFNQVLEHMMFWKEDKNFDVEHHLHHVALPKPARVRELLMYVSREHGRLLDRAMPLWECHVIEGIQPETEGSPERFALYFKIHHSLVDGIAAMRLVKKSLSQSPNEPVTLPIWSLMAHHRNQIDAIFPKERSALRILKEQVSTIKPVFTELLNNFKNYNDDSYVSTFDAPRSILNRRISASRRIAAQSYDIKRFNDIAERINISKNDVVLAVCSGAIRRYLISMDALPSKPLIAFVPMSLRTDDSIAGNQLSFVLANLGTHLDDPLSRIKLIHRSMNNSKRRFRRMNQAQVINYSIVSYAWEGINLATDLFPKKQAFNLIISNVPGSEKPLYWNGARLESLYPASIVFNGQAMNITLASYLDKMEFGITACSKALPHVQDMLMLIEEELQLLESVSKELEFNGITVKDKSEKKLKKLAP